(19) United States
(12) Patent Application Publication
(10) Pub. No.: US 2026/0308140 A1
(43) Pub. Date: Oct. 8, 2026

(54) Cis-8-(3,5-Difluorophenyl)-8-(dimethylamino)-1,3-diazaspiro[4.5]decan-2-one derivatives

(71) Applicant: Gruenenthal GmbH, Aachen (DE)

(72) Inventors: Sebastian PEIL, Aachen (DE); Inna SLYNKO, Aachen (DE); Ingo KONETZKI, Aachen (DE); Nikolay SITNIKOV, Aachen (DE); Philipp BARBIE, Aachen (DE); Mauro MARIGO, Aachen (DE); Martin PETTERSSON, Aachen (DE); David ST. JEAN, Aachen (DE)

(73) Assignee: Gruenenthal GmbH, Aachen (DE)

(21) Appl. No.: 19/478,558

(22) PCT Filed: Apr. 25, 2024
(86) PCT No.: PCT/EP2024/061353

§ 371 Date: Oct. 24, 2025

Related U.S. Application Data
(60) Provisional application No. 63/461,659, filed on Apr. 25, 2023.

(30) Foreign Application Priority Data
Apr. 25, 2023 (EP) 23169716.0

(51) Int. Cl. C07D 401/04; A61K 31/437; A61K 31/4439; A61K 31/497; A61K 31/501; A61K 31/506; A61P 25/04; C07D 403/04; C07D 405/14; C07D 471/04

(52) U.S. Cl. CPC C07D 401/04; A61K 31/437; A61K 31/4439; A61K 31/497; A61K 31/501; A61K 31/506; A61P 25/04; C07D 403/04; C07D 405/14; C07D 471/04

(57) ABSTRACT

The invention relates to cis-8-(3,5-difluorophenyl)-8-(dimethylamino)-1,3-diazaspiro[4.5]-decan-2-one derivatives, their preparation and use in medicine, particularly in various neurological disorders, including but not limited to pain, neurodegenerative disorders, neuroinflammatory disorders, neuropsychiatric disorders, substance abuse/dependence.

Priority is claimed of European patent application no. 23169716.0 that was filed on Apr. 25, 2023 and of U.S. provisional patent application No. 63/461,659 that was filed on Apr. 25, 2023.

The invention relates to cis-8-(3,5-difluorophenyl)-8-(alkylamino)-1,3-diazaspiro[4.5]-decan-2-one derivatives, their preparation and use in medicine, particularly in various neurological disorders, including but not limited to pain, neurodegenerative disorders, neuroinflammatory disorders, neuropsychiatric disorders, substance abuse/dependence.

Opioid receptors are a group of Gi/o protein-coupled receptors which are widely distributed in the human body. The opioid receptors are currently subdivided into four major classes, i.e. the three classical opioid receptors mu-opioid (MOP) receptor, kappa-opioid (KOP) receptor, and delta-opioid (DOP) receptor as well as the opioid receptor-like (ORL-1) receptor, which was more recently discovered based on its high homology with said classical opioid receptors. After identification of the endogenous ligand of the ORL-1 receptor, known as nociceptin/orphanin FQ, a highly basic 17 amino acid peptide isolated from tissue extracts in 1995, the ORL-1 receptor was renamed “nociceptin opioid peptide receptor” and abbreviated as “NOP-receptor”.

The classical opioid receptors (MOP, KOP and DOP) as well as the NOP receptor are widely distributed/expressed in the human body, including in the brain, the spinal cord, on peripheral sensory neurons and the intestinal tract, wherein the distribution pattern differs between the different receptor classes.

Nociceptin acts at the molecular and cellular level in very much the same way as opioids. However, its pharmacological effects sometimes differ from, and even oppose those of opioids. NOP-receptor activation translates into a complex pharmacology of pain modulation, which, depending on route of administration, pain model and species involved, leads to either pronociceptive or antinociceptive activity. Furthermore, the NOP receptor system is upregulated under conditions of chronic pain. Systemic administration of selective NOP receptor agonists was found to exert a potent and efficacious analgesia in non-human primate models of acute and inflammatory pain in the absence of side effects. The activation of NOP receptors has been demonstrated to be devoid of reinforcing effects but to inhibit opioid-mediated reward in rodents and non-human primates (Review: Schroeder et al, Br J Pharmacol 2014; 171 (16): 3777-3800, and references therein).

Besides the involvement of the NOP receptor in nociception, results from preclinical experiments suggest that NOP receptor agonists might be useful inter alia in the treatment of neuropsychiatric disorders (Witkin et al, Pharmacology & Therapeutics, 141 (2014) 283-299; Jenck et al., Proc. Natl. Acad. Sci. USA 94, 1997, 14854-14858).

Strong opioids acting at the MOP receptor site are widely used to treat moderate to severe acute and chronic pain. However, the therapeutic window of strong opioids is limited by severe side effects such as nausea and vomiting, constipation, dizziness, somnolence, respiratory depression, physical dependence and abuse. Furthermore, it is known that MOP receptor agonists show only reduced effectiveness under conditions of chronic and neuropathic pain.

Alternatively, the delta opioid receptor (DOP) has been a target of interest for a number of years as a potential for treatment of pain, as well as for anxiety and depression. Although, selective DOP ligands have generally failed due to lack of efficacy, notable safety liabilities, such as convulsions, effects on locomotor activity/coordination, tolerance, headache and gastrointestinal discomfort, have been reported in preclinical or clinical studies (Broom et al, 2002; Hudzik et al, 2014; Spahn & Stein, 2017). These safety related effects have been shown to be differentiated across the selective DOP ligands, with emerging evidence suggesting diverse downstream signaling pathways, receptor phosphorylation, receptor trafficking and selectivity add complexity to understanding the biological and pharmacological responses of DOP ligands (Broon et al, 2002; Pradhan et al, 2012; Mann et al, 2020; Quirion et al, 2020).

It is known that some of the above mentioned side-effects of strong opioids are mediated by activation of classic opioid-receptors within the central nervous system. Furthermore, peripheral opioid receptors, when activated, can inhibit transmission of nociceptive signals shown in both, clinical and animal studies (Gupta et al., 2001; Kalso et al., 2002; Stein et al., 2003; Zollner et al., 2008).

Thus, to avoid CNS-mediated adverse effects after systemic administration, one approach has been to provide peripherally restricted opioid receptor ligands that do not easily cross the blood-brain barrier and therefore distribute poorly to the central nervous system (see for instance WO 2015/192039). Such peripherally acting compounds might combine effective analgesia with limited side-effects.

Another approach has been to provide compounds which interact with both the NOP receptor and the MOP receptor. Such compounds have for instance been described in WO 2004/043967, WO 2012/013343 and WO 2009/118168.

A further approach has been to provide multi-opioid receptor analgesics that modulate more than one of the opioid receptor subtypes to provide additive or synergistic analgesia and/or reduced side effects like abuse liability or tolerance.

On the one hand, it would be desirable to provide analgesics that selectively act on the NOP receptor system but less pronounced on the classic opioid receptor system, whereas it would be desirable to distinguish between central nervous activity and peripheral nervous activity.

8-(Alkylamino)-1,3-diazaspiro[4.5]decan-2-one derivatives are known from e.g. WO 2017 121646, WO 2017 121647, WO 2017 121648, WO 2017 121649, WO 2017 121650, WO 2019 012037.

There is a need for medicaments which are effective in the treatment of pain and which have advantages compared to the compounds of the prior art. Where possible, such medicaments should contain such a small dose of active ingredient that satisfactory pain therapy can be ensured without the occurrence of intolerable treatment-emergent adverse events.

It is an object of the invention to provide pharmacologically active compounds, preferably analgesics that have advantages compared to the prior art.

This object has been achieved by the subject-matter of the patent claims.

The invention relates to a compound according to general formula (I)

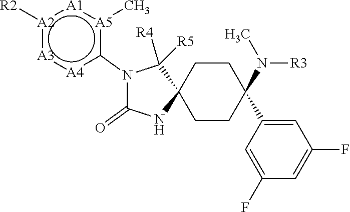

wherein
A1 represents N or CR1;
A2 represents N or C;
A3 represents N or CH;
A4 represents N or CH;
A5 represents N or C;
with the proviso that one or two of A1, A2, A3, A4 and A5 mean N, whereas the remaining three or four of A1, A2, A3, A4, and A5 do not mean N;
R1 represents —H, —CH₃ or ═O;
R2 represents
   —C₁-C₆-alkyl, linear or branched, saturated or unsaturated, unsubstituted or substituted with one, two, three or four substituents independently selected from —F and —OH;
   —C₃-C₇-heterocycloalkyl, saturated or unsaturated, unsubstituted or substituted with one, two, three or four substituents independently selected from —F, —OH and —CH₃;
   —S(O)₂—C₁-C₄-alkyl, linear, branched or cyclic, unsubstituted or substituted with one, two or three substituents —F;
   —O—C₁-C₆-alkyl, linear or branched, saturated or unsaturated, unsubstituted or substituted with one, two, three or four substituents independently selected from —F, —OH and —OCH₃;
   —O—C₃-C₈-cycloalkyl, saturated or unsaturated, unsubstituted or substituted with one, two, three or four substituents independently selected from —F, —OH and —CH₃; or
   —O—C₃-C₇-heterocycloalkyl, saturated or unsaturated, unsubstituted or substituted with one, two, three or four substituents independently selected from —F and —OH;
or R1 and R2 together with A1 and A2 form a ring and mean —CH═CN—NH—;
R3 represents —H or —CH₃; and
R4 and R5 both represent either each —H or together mean ═O;
or a physiologically acceptable salt thereof.

The compounds according to the invention have in common a new structural motif which is not known from the prior art (e.g.). The structural motif is characterized by the 3,5-difluorophenyl moiety and the methyl group at A5.

As demonstrated by the comparative experimental data, the new structural motif has the effect that consistently higher selectivity with respect to DOP is achieved.

According to the invention, unless expressly stated otherwise, “—C₁-C₆-alkyl” and any other alkyl residues can be saturated linear or branched. Linear saturated alkyl includes methyl, ethyl, n-propyl, n-butyl, n-pentyl and n-hexyl. Examples of branched saturated alkyl include but are not limited to isopropyl, sec-butyl, and tert-butyl.

According to the invention, “C₃-C₈-cycloalkyl” means a non-aromatic, monocyclic or bicyclic moiety comprising 3 to 8 ring carbon atoms but no heteroatoms in the ring. Examples of preferred saturated C₃-C₈-cycloalkyls according to the invention include but are not limited to cyclopropane, cyclobutane, cyclopentane, cyclohexane, cycloheptane, cyclooctane. Examples of preferred unsaturated C₃-C₈-cycloalkyls according to the invention include but are not limited to cyclopropene, cyclobutene, cyclopentene, cyclopentadiene, cyclohexene, 1,3-cyclohexadiene, and 1,4-cyclohexadiene.

According to the invention, “C₃-C₇-heterocycloalkyl” means a non-aromatic, monocyclic or bicyclic moiety comprising 4 to 8 ring atoms, wherein each cycle comprises independently of one another 1, 2, 3, 4 or more heteroatoms independently of one another selected from the group consisting of nitrogen, oxygen and sulfur, whereas sulfur may be oxidized (S(═O) or (S(═O)₂), whereas the remaining ring atoms are carbon atoms, and whereas bicyclic systems may share common heteroatom(s). Examples of preferred saturated C₃-C₇-heterocycloalkyls according to the invention include but are not limited to azetidine, pyrrolidine, oxetane and tetrahydrofuran.

In preferred embodiments, R4 and R5 both represent each —H.

In preferred embodiments, one of A1, A2, A3, A4 and A5 represents N, whereas the remaining four of A1, A2, A3, A4, and A5 do not represent N.

In other preferred embodiments, two of A1, A2, A3, A4 and A5 represent N, whereas the remaining three of A1, A2, A3, A4, and A5 do not represent N.

In preferred embodiments, A1 represents CR1; A2 represents C; A3 represents CH; A4 represents N; and A5 represents C, according to general formula (II-A):

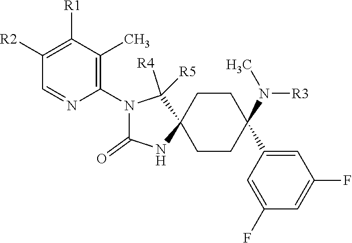

When R3 represents —CH₃ and R4 and R5 each represent —H, this structural motif is realized by Examples EX-I-01, EX-I-03, EX-I-04, EX-I-05, EX-I-07, EX-I-09, EX-I-10, EX-I-11, EX-I-24, and EX-I-26.

When R3, R4 and R5 each represent —H, this structural motif is realized by Examples EX-I-06 and EX-I-25.

When R3 represents —CH₃ and R4 and R5 together mean ═O, this structural motif is realized by Example EX-I-28.

In preferred embodiments, A1 represents N; A2 represents C; A3 represents CH; A4 represents CH; and A5 represents C, according to general formula (II-B):

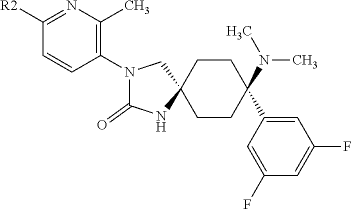

This structural motif is realized by Examples EX-I-12, EX-I-13, EX-I-14, and EX-I-17.

In preferred embodiments, A1 represents CR1; A2 represents C; A3 represents N; A4 represents N; and A5 represents C, according to general formula (II-C):

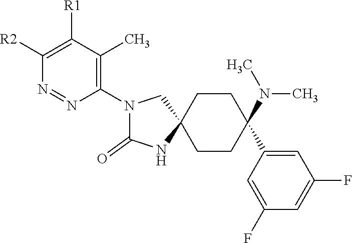

This structural motif is realized by Examples EX-I-08 and EX-I-27.

In preferred embodiments, A1 represents N; A2 represents C; A3 represents N; A4 represents CH; and A5 represents C, according to general formula (II-D):

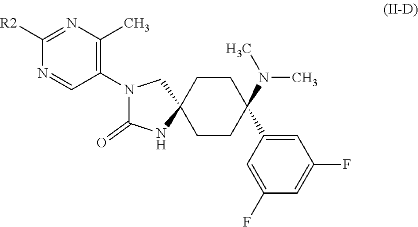

This structural motif is realized by Examples EX-I-02, EX-I-15, EX-I-16, EX-I-18, EX-I-21, and EX-I-22.

In preferred embodiments, A1 represents CR1 with R1 meaning ═O; A2 represents N; A3 represents CH; A4 represents CH; and A5 represents C, according to general formula (II-E):

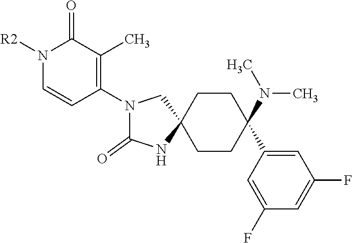

This structural motif is realized by Example EX-I-19.

In preferred embodiments, A1 represents N; A2 represents C; A3 represents CH; A4 represents N; and A5 represents C, according to general formula (II-F):

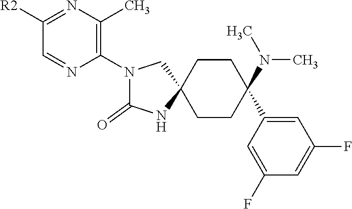

This structural motif is realized by Example EX-I-20.

In preferred embodiments, A1 represents CR1 with R1 meaning ═O; A2 represents C; A3 represents CH; A4 represents CH; and A5 represents N, according to general formula (II-G):

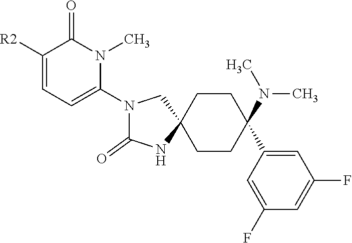

This structural motif is realized by Example EX-I-23.

In preferred embodiments, R1 (if present) represents —H, or —CH₃.

In preferred embodiments, R2 represents —C₁-C₃-alkyl, linear or branched, saturated, unsubstituted or substituted with one, two, three, or four substituents independently selected from —F and —OH; preferably —CH₃, —CF₃, —C(CH₃)₂OH, or —C(CH₃)(OH)CF₃.

In preferred embodiments, R2 represents —C₄-heterocycloalkyl, saturated, unsubstituted or substituted with one, two, or three substituents independently selected from —F, —OH and —OCH₃; preferably 3-fluorooxetan-3-yl.

In preferred embodiments, R2 represents —S(O)₂—C₁-C₂-alkyl, linear, unsubstituted; preferably —S(O)₂CH₃ or —S(O)₂CH₂CH₃.

In preferred embodiments, R2 represents —O—C₁-C₂-alkyl, saturated, unsubstituted or substituted with one, two, or three substituents independently selected from —F, —OH and —OCH₃; preferably —OCH₃, —O—CH₂CH₂—OCH₃, —OCHF₂, —OCH₂CHF₂, —OCF₂CH₃, or —OCF₃.

In preferred embodiments, R2 represents —O—C₃-C₄-cycloalkyl, saturated, unsubstituted or substituted with one or two substituents independently selected from —OH and —CH₃; preferably —O-cyclopropyl or —O-(3-hydroxy-3-methyl)-cyclobutyl (i.e., 3-hydroxy-3-methylcyclobutoxy).

In preferred embodiments, R2 represents —O—C₄-heterocycloalkyl, saturated, unsubstituted; preferably —O-oxetan-3-yl.

In preferred embodiments, R2 represents —CH₃, according to general formula (III-A):

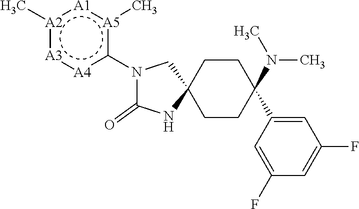

This structural motif is realized by Examples EX-I-13 and EX-I-19.

In preferred embodiments, R2 represents —CF₃, according to general formula (III-B):

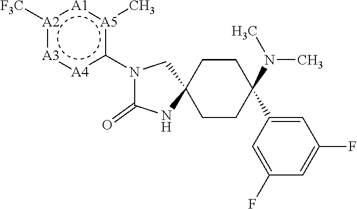

This structural motif is realized by Example EX-I-12.

In preferred embodiments, R2 represents —C(CH₃)₂OH, according to general formula (III-C):

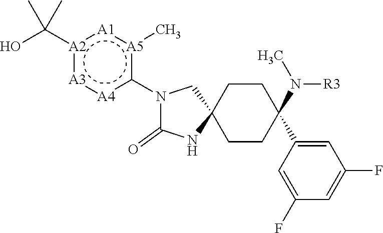

When R3 represents —CH₃, this structural motif is realized by Examples EX-I-03, EX-I-20 and EX-I-22. When R3 represents —H, this structural motif is realized by Example EX-I-06.

In preferred embodiments, R2 represents —S(O)₂CH₃, according to general formula (III-D):

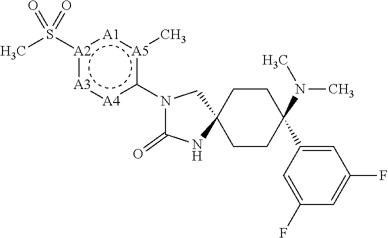

This structural motif is realized by Examples EX-I-10 and EX-I-17.

In preferred embodiments, R2 represents —S(O)₂CH₂CH₃, according to general formula (III-E):

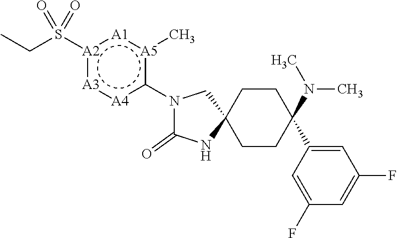

This structural motif is realized by Example EX-I-11.

In preferred embodiments, R2 represents —OCH₃, according to general formula (III-F):

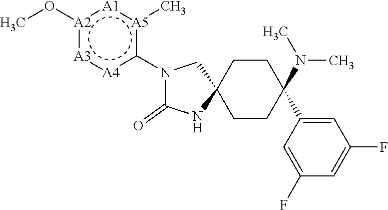

This structural motif is realized by Example EX-I-02.

In preferred embodiments, R2 represents —OCHF₂, according to general formula (III-G):

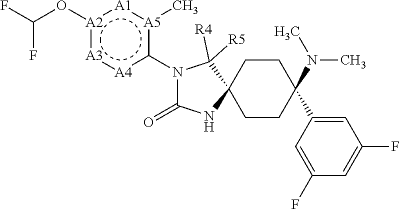

When R4 and R5 each mean —H, this structural motif is realized by Example EX-I-04.

When R4 and R5 together mean ═O, this structural motif is realized by Example EX-I-28.

In preferred embodiments, R2 represents —OCF₃, according to general formula (III-H):

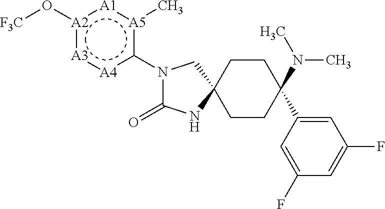

This structural motif is realized by Examples EX-I-07, EX-I-08, and EX-I-14.

In preferred embodiments, R2 represents —OCH₂CH₂OCH₃, according to general formula (IIII):

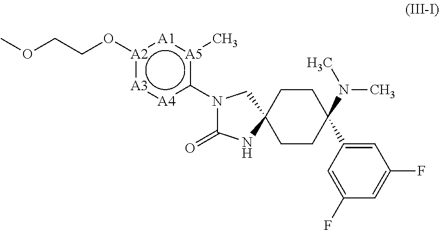

This structural motif is realized by Example EX-I-16.

In preferred embodiments, R2 represents —O-cyclopropyl, according to general formula (III-J):

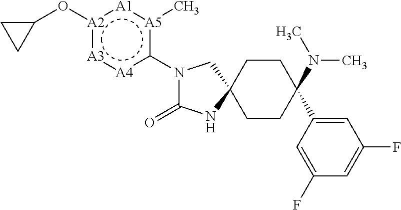

This structural motif is realized by Examples EX-I-01, EX-I-18 and EX-I-25.

In preferred embodiments, R2 represents 3-hydroxy-3-methylcyclobutoxy, according to general formula (III-K):

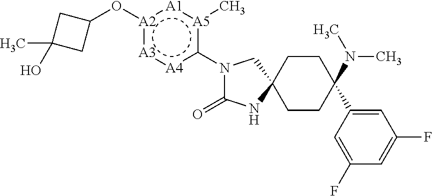

This structural motif is realized by Example EX-I-09.

In preferred embodiments, R2 represents oxetan-3-yloxy, according to general formula (III-L):

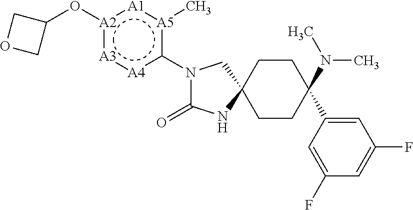

This structural motif is realized by Example EX-I-15.

In preferred embodiments, R1 and R2 together with A1 and A2 form a ring and mean —CH═CN—NH—, according to general formula (III-M):

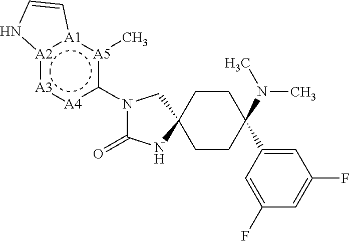

This structural motif is realized by Example EX-I-05.

In preferred embodiments, R2 represents 3-fluorooxetan-3-yl, according to general formula (III-N):

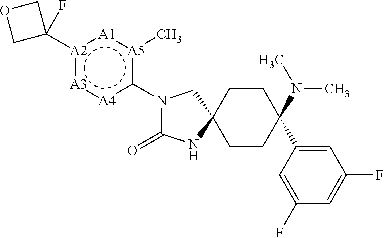

This structural motif is realized by Example EX-I-21.

In preferred embodiments, R2 represents —OCHF₂, according to general formula (III-O):

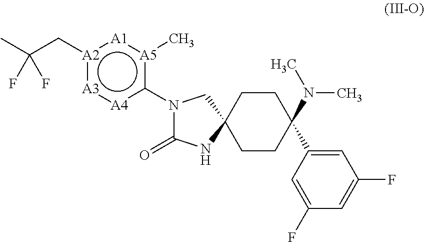

This structural motif is realized by Example EX-I-23.

In preferred embodiments, R2 represents —C(CH₃)(OH)CF₃, according to general formula (III-P):

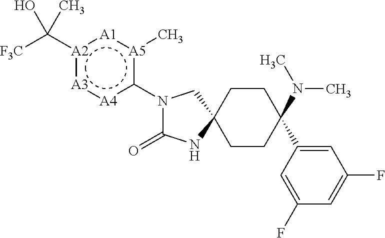

This structural motif is realized by Example EX-I-24. As R2 is chiral, two stereoisomers exist.

In preferred embodiments, the compound according to general formula (III-P) has R2 in R configuration. In other preferred embodiments, the compound according to general formula (III-P) has R2 in S configuration.

In preferred embodiments, R2 represents —OCH₂CHF₂, according to general formula (III-Q):

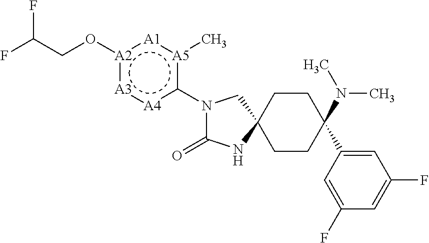

This structural motif is realized by Examples EX-I-26 and EX-I-27.

In preferred embodiments, R3 represents —H.

In other preferred embodiments, R3 represents —CH₃.

In preferred embodiments, R4 and R5 each mean —H.

In other preferred embodiments, R4 and R5 together represent ═O.

In particularly preferred embodiments, the compound according to the invention is selected from the group consisting of:

| | | |
|---|---|---|
| EX-I- 01 | 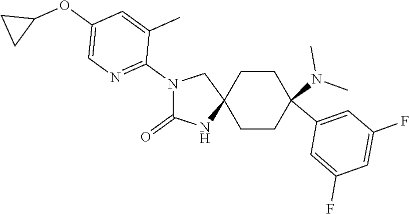 [SMILES: Cc1cc(OC2CC2)cnc1N1C[C@]2(CC[C@](c3cc(F)cc(F)c3)(N(C)C)CC2)NC1=O] | cis-3-(5-cyclopropoxy-3- methylpyridin-2-yl)-8- (3,5-difluorophenyl)-8- (dimethylamino)-1,3- diazaspiro[4.5]decan-2-one |
| EX-I- 02 | 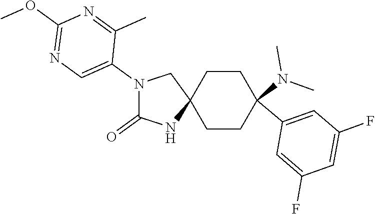 [SMILES: COc1ncc(N2C[C@]3(CC[C@](c4cc(F)cc(F)c4)(N(C)C)CC3)NC2=O)c(C)n1] | cis-8-(3,5-difluoro- phenyl)-8-(dimethyl- amino)-3-(2-methoxy-4- methylpyrimidin-5-yl)- 1,3-diazaspiro [4.5]decan-2-one |
| EX-I- 03 | 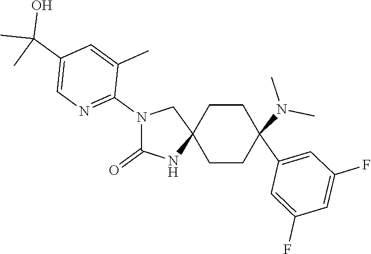 [SMILES: Cc1cc(C(C)(C)O)cnc1N1C[C@]2(CC[C@](c3cc(F)cc(F)c3)(N(C)C)CC2)NC1=O] | cis-8-(3,5-difluoro- phenyl)-8-(dimethyl- amino)-3-(5-(2-hydroxy- propan-2-yl)-3- methylpyridin-2-yl)-1,3- diazaspiro[4.5]decan-2- one |
| EX-I- 04 | 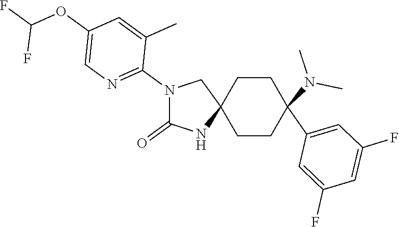 [SMILES: Cc1cc(OC(F)F)cnc1N1C[C@]2(CC[C@](c3cc(F)cc(F)c3)(N(C)C)CC2)NC1=O] | cis-3-(5-(difluorometh- oxy)-3-methylpyridin-2- yl)-8-(3,5-difluoro- phenyl)-8-(dimethyl- amino)-1,3-diazaspiro [4.5]decan-2-one |
| EX-I- 05 | 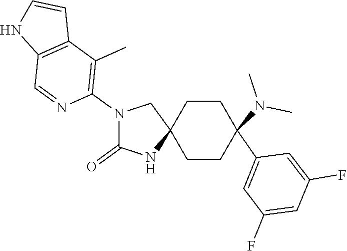 [SMILES: Cc1c(N2C[C@]3(CC[C@](c4cc(F)cc(F)c4)(N(C)C)CC3)NC2=O)ncc2[nH]ccc12] | cis-8-(3,5-difluoro- phenyl)-8-(dimethyl- amino)-3-(4-methyl-1H- pyrrolo[2,3-c]pyridin-5- yl)-1,3-diazaspiro [4.5]decan-2-one |
| EX-I- 06 | 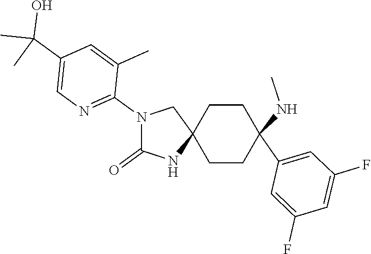 [SMILES: CN[C@]1(c2cc(F)cc(F)c2)CC[C@]2(CC1)CN(c1ncc(C(C)(C)O)cc1C)C(=O)N2] | cis-8-(3,5-difluoro- phenyl)-3-(5-(2-hydroxy- propan-2-yl)-3- methylpyridin-2-yl)-8- (methylamino)-1,3- diazaspiro[4.5]decan-2-one |
| EX-I- 07 | 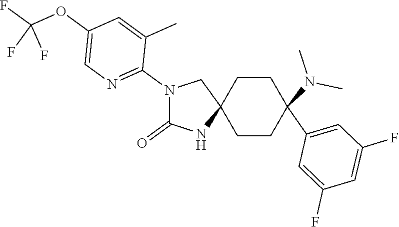 [SMILES: Cc1cc(OC(F)(F)F)cnc1N1C[C@]2(CC[C@](c3cc(F)cc(F)c3)(N(C)C)CC2)NC1=O] | cis-8-(3,5-difluoro- phenyl)-8-(dimethyl- amino)-3-(3-methyl-5-(tri- fluoromethoxy)pyridin- 2-yl)-1,3-diazaspiro- [4.5]decan-2-one |
| EX-I- 08 | 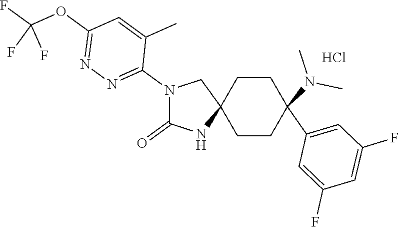 [SMILES: Cc1cc(OC(F)(F)F)nnc1N1C[C@]2(CC[C@](c3cc(F)cc(F)c3)(N(C)C)CC2)NC1=O ; Cl] | cis-8-(3,5-difluoro- phenyl)-8-(dimethyl- amino)-3-(4-methyl-6-(tri- fluoromethoxy) pyridazin-3-yl)-1,3- diazaspiro[4.5]decan-2-one hydrochloride |
| EX-I- 09 | 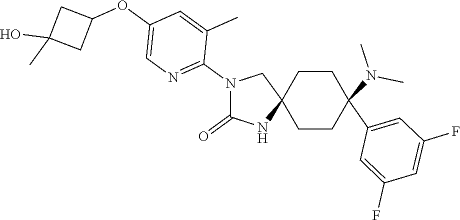 [SMILES: Cc1cc(OC2CC(C)(O)C2)cnc1N1C[C@]2(CC[C@](c3cc(F)cc(F)c3)(N(C)C)CC2)NC1=O] | cis-8-(3,5-difluoro- phenyl)-8-(dimethyl- amino)-3-(5-(3-hydroxy- 3-methylcyclobutoxy)-3- methylpyridin-2-yl)-1,3- diazaspiro[4.5]decan-2- one |
| EX-I- 10 | 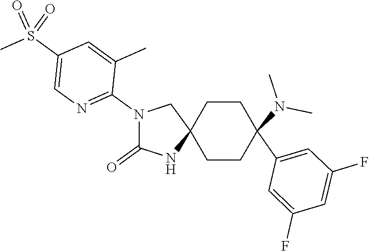 [SMILES: Cc1cc(S(C)(=O)=O)cnc1N1C[C@]2(CC[C@](c3cc(F)cc(F)c3)(N(C)C)CC2)NC1=O] | cis-8-(3,5-difluoro- phenyl)-8-(dimethyl- amino)-3-(3-methyl-5- (methylsulfonyl)pyridin- 2-yl)-1,3- diazaspiro[4.5]decan-2-one |
| EX-I- 11 | 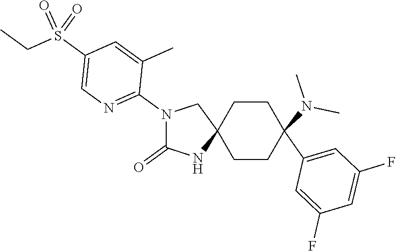 [SMILES: CCS(=O)(=O)c1cnc(N2C[C@]3(CC[C@](c4cc(F)cc(F)c4)(N(C)C)CC3)NC2=O)c(C)c1] | cis-8-(3,5-difluoro- phenyl)-8-(dimethyl- amino)-3-(5-(ethyl- sulfonyl)-3-methylpyridin- 2-yl)-1,3- diazaspiro[4.5]decan-2-one |
| EX-I- 12 | 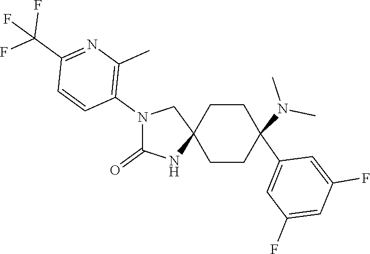 [SMILES: Cc1nc(C(F)(F)F)ccc1N1C[C@]2(CC[C@](c3cc(F)cc(F)c3)(N(C)C)CC2)NC1=O] | cis-8-(3,5-difluoro- phenyl)-8-(dimethyl- amino)-3-(2-methyl-6-(tri- fluoromethyl)pyridin-3- yl)-1,3- diazaspiro[4.5]decan-2-one |
| EX-I- 13 | 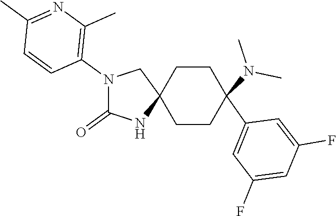 [SMILES: Cc1ccc(N2C[C@]3(CC[C@](c4cc(F)cc(F)c4)(N(C)C)CC3)NC2=O)c(C)n1] | cis-8-(3,5-difluoro- phenyl)-8-(dimethyl- amino)-3-(2,6- dimethylpyridin-3-yl)-1,3- diazaspiro[4.5]decan-2-one |
| EX-I- 14 | 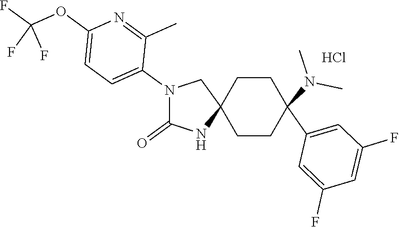 [SMILES: Cc1nc(OC(F)(F)F)ccc1N1C[C@]2(CC[C@](c3cc(F)cc(F)c3)(N(C)C)CC2)NC1=O ; Cl] | cis-8-(3,5-difluoro- phenyl)-8-(dimethyl- amino)-3-(2-methyl-6-(tri- fluoromethoxy)pyridin- 3-yl)-1,3- diazaspiro[4.5]decan-2-one hydrochloride |
| EX-I- 15 | 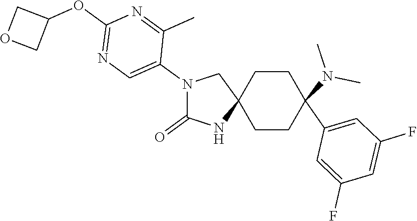 [SMILES: Cc1nc(OC2COC2)ncc1N1C[C@]2(CC[C@](c3cc(F)cc(F)c3)(N(C)C)CC2)NC1=O] | cis-8-(3,5-difluoro- phenyl)-8-(dimethyl- amino)-3-(4-methyl-2- (oxetan-3-yloxy)pyrimidin- 5-yl)-1,3-diazaspiro [4.5]decan-2-one |
| EX-I- 16 | 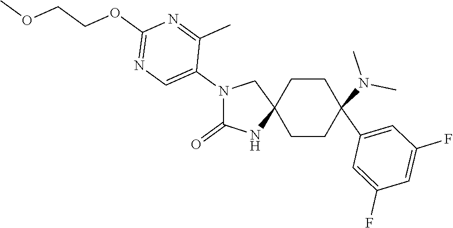 [SMILES: COCCOc1ncc(N2C[C@]3(CC[C@](c4cc(F)cc(F)c4)(N(C)C)CC3)NC2=O)c(C)n1] | cis-8-(3,5-difluoro- phenyl)-8-(dimethyl- amino)-3-(2-(2-methoxy- ethoxy)-4-methylpyrimidin- 5-yl)-1,3-diazaspiro [4.5]decan-2-one |
| EX-I- 17 | 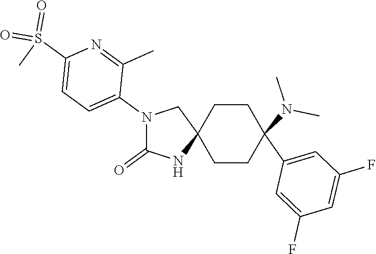 [SMILES: Cc1nc(S(C)(=O)=O)ccc1N1C[C@]2(CC[C@](c3cc(F)cc(F)c3)(N(C)C)CC2)NC1=O] | cis-8-(3,5-difluoro- phenyl)-8-(dimethyl- amino)-3-(2-methyl-6- (methylsulfonyl)pyridin- 3-yl)-1,3- diazaspiro[4.5]decan-2-one |
| EX-I- 18 | 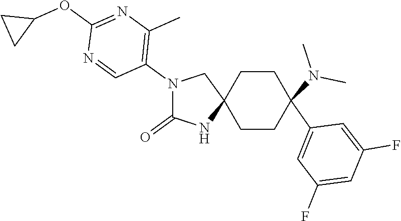 [SMILES: Cc1nc(OC2CC2)ncc1N1C[C@]2(CC[C@](c3cc(F)cc(F)c3)(N(C)C)CC2)NC1=O] | cis-3-(2-cyclopropoxy-4- methylpyrimidin-5-yl)-8- (3,5-difluorophenyl)-8- (dimethylamino)-1,3- diazaspiro[4.5]decan-2-one |
| EX-I- 19 | 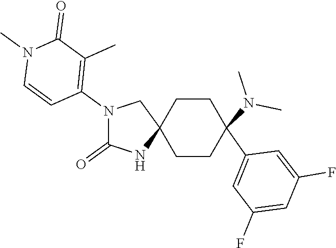 [SMILES: Cc1c(N2C[C@]3(CC[C@](c4cc(F)cc(F)c4)(N(C)C)CC3)NC2=O)ccn(C)c1=O] | cis-8-(3,5-difluoro- phenyl)-3-(1,3-dimethyl- 2-oxo-1,2-dihydro- pyridin-4-yl)-8- (dimethylamino)-1,3- diazaspiro[4.5]decan-2-one |
| EX-I- 20 | 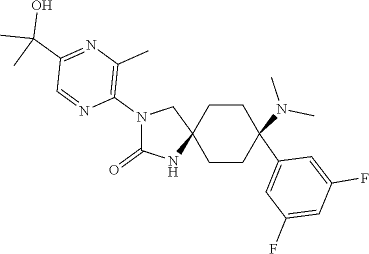 [SMILES: Cc1nc(C(C)(C)O)cnc1N1C[C@]2(CC[C@](c3cc(F)cc(F)c3)(N(C)C)CC2)NC1=O] | cis-8-(3,5-difluoro- phenyl)-8-(dimethyl- amino)-3-(5-(2-hydroxy- propan-2-yl)-3- methylpyrazin-2-yl)-1,3- diazaspiro[4.5]decan-2- one |
| EX-I- 21 | 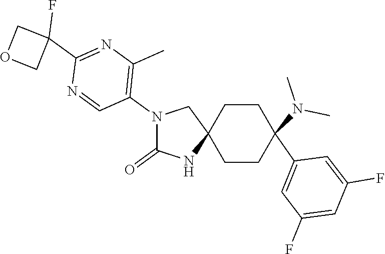 [SMILES: Cc1nc(C2(F)COC2)ncc1N1C[C@]2(CC[C@](c3cc(F)cc(F)c3)(N(C)C)CC2)NC1=O] | cis-8-(3,5-difluoro- phenyl)-8-(dimethyl- amino)-3-(2-(3-fluorooxetan- 3-yl)-4-methylpyrimidin- 5-yl)-1,3-diazaspiro [4.5]decan-2-one |
| EX-I- 22 | 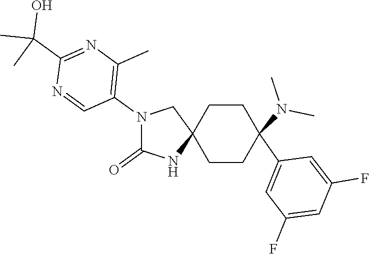 [SMILES: Cc1nc(C(C)(C)O)ncc1N1C[C@]2(CC[C@](c3cc(F)cc(F)c3)(N(C)C)CC2)NC1=O] | cis-8-(3,5-difluoro- phenyl)-8-(dimethyl- amino)-3-(2-(2-hydroxy- propan-2-yl)-4- methylpyrimidin-5-yl)- 1,3-diazaspiro [4.5]decan-2-one |
| EX-I- 23 | 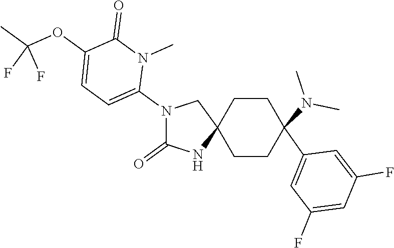 [SMILES: Cn1c(N2C[C@]3(CC[C@](c4cc(F)cc(F)c4)(N(C)C)CC3)NC2=O)ccc(OC(C)(F)F)c1=O] | cis-3-(5-(1,1-difluoroethoxy)- 1-methyl-6-oxo- 1,6-dihydropyridin-2-yl)- 8-(3,5-difluorophenyl)-8- (dimethylamino)-1,3- diazaspiro[4.5]decan-2-one |
| EX-I- 24 | 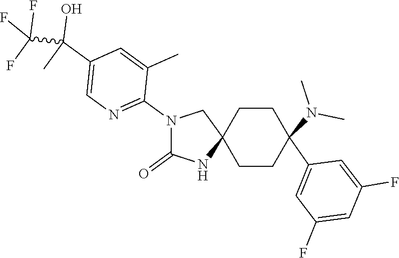 [SMILES: Cc1cc(C(C)(O)C(F)(F)F)cnc1N1C[C@]2(CC[C@](c3cc(F)cc(F)c3)(N(C)C)CC2)NC1=O] | cis-8-(3,5-difluoro- phenyl)-8-(dimethyl- amino)-3-(3-methyl-5- ((S)-1,1,1-trifluoro-2- hydroxypropan-2-yl) pyridin-2-yl)-1,3- diazaspiro[4.5]decan-2-one |
| EX-I- 25 | 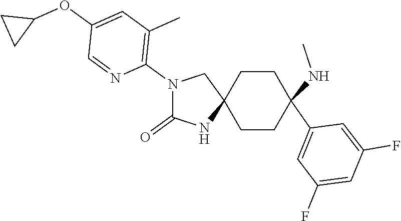 [SMILES: CN[C@]1(c2cc(F)cc(F)c2)CC[C@]2(CC1)CN(c1ncc(OC3CC3)cc1C)C(=O)N2] | cis-3-(5-cyclopropoxy-3- methylpyridin-2-yl)-8- (3,5-difluorophenyl)-8- (methylamino)-1,3- diazaspiro[4.5]decan-2-one |
| EX-I- 26 | 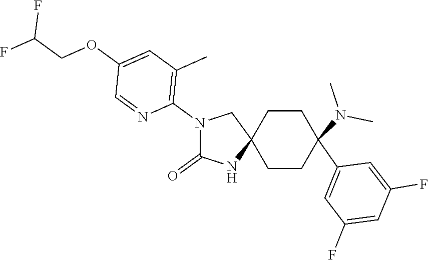 [SMILES: Cc1cc(OCC(F)F)cnc1N1C[C@]2(CC[C@](c3cc(F)cc(F)c3)(N(C)C)CC2)NC1=O] | cis1-3-(5-(2,2-difluoro- ethoxy)-3-methylpyridin- 2-yl)-8-(3,5-difluoro- phenyl)-8-(dimethyl- amino)-1,3- diazaspiro[4.5]decan-2-one |
| EX-I- 27 | 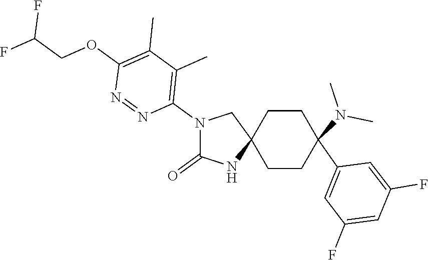 [SMILES: Cc1c(OCC(F)F)nnc(N2C[C@]3(CC[C@](c4cc(F)cc(F)c4)(N(C)C)CC3)NC2=O)c1C] | cis-3-(6-(2,2-difluoroethoxy)- 4,5-dimethylpyridazin- 3-yl)-8-(3,5- difluorophenyl)-8- (dimethylamino)-1,3- diazaspiro[4.5]decan-2-one |
| EX-I- 28 | 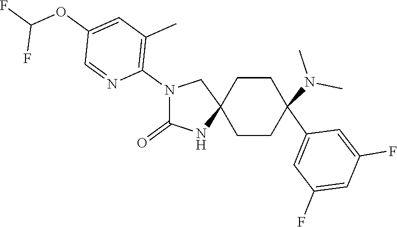 [SMILES: Cc1cc(OC(F)F)cnc1N1C[C@]2(CC[C@](c3cc(F)cc(F)c3)(N(C)C)CC2)NC1=O] | cis-3-(5-(difluorometh- oxy)-3-methylpyridin-2- yl)-8-(3,5-difluoro- phenyl)-8-(dimethyl- amino)-1,3- diazaspiro[4.5]decane-2,4- dione |

and the physiologically acceptable salts thereof.

In a preferred embodiment, the compounds according to the invention are in the form of the free bases.

In another preferred embodiment, the compounds according to the invention are in the form of the physiologically acceptable salts.

For the purposes of the description, a “salt” is to be understood as being any form of the compound in which it assumes an ionic form or is charged and is coupled with a counter-ion (a cation or anion) or is in solution. The term is also to be understood as meaning complexes of the compound with other molecules and ions, in particular complexes which are associated via ionic interactions. Preferred salts are physiologically acceptable, in particular physiologically acceptable salts with anions or acids or also a salt formed with a physiologically acceptable acid.

Physiologically acceptable salts with anions or acids are salts of the particular compound in question with inorganic or organic acids which are physiologically acceptable, in particular when used in humans and/or mammals. Examples of physiologically acceptable salts of particular acids include but are not limited to salts of hydrochloric acid, sulfuric acid, and acetic acid.

The invention also includes enantiomers or diastereomers, as racemates or in any enantiomeric excess and diastereomeric excess, respectively.

The invention also includes isotopic isomers of a compound according to the invention, wherein at least one atom of the compound is replaced by an isotope of the respective atom which is different from the naturally predominantly occurring isotope, as well as any mixtures of isotopic isomers of such a compound. Preferred isotopes are ²H (deuterium), 3H (tritium), ¹³C and ¹⁴C.

A further aspect of the invention relates to the compounds according to the invention as medicaments.

A further aspect of the invention relates to the compounds according to the invention for use in the treatment of pain. A further aspect of the invention relates to a method of treating pain comprising the administration of a pain alleviating amount of a compound according to the invention to a subject in need thereof, preferably to a human. The pain is preferably acute or chronic. The pain is preferably nociceptive or neuropathic.

A further aspect of the invention relates to the compounds according to the invention for use in the treatment of neurodegenerative disorders, neuroinflammatory disorders, neuropsychiatric disorders, and substance abuse/dependence. A further aspect of the invention relates to a method of treating any one of the aforementioned disorders, diseases or conditions comprising the administration of a therapeutically effective amount of a compound according to the invention to a subject in need thereof, preferably to a human.

Another aspect of the invention relates to a pharmaceutical composition which contains a physiologically acceptable carrier and at least one compound according to the invention.

Preferably, the composition according to the invention is solid, liquid or pasty; and/or contains the compound according to the invention in an amount of from 0.001 to 99 wt. %, preferably from 1.0 to 70 wt. %, based on the total weight of the composition.

The pharmaceutical composition according to the invention can optionally contain suitable additives and/or auxiliary substances and/or optionally further active ingredients.

Examples of suitable physiologically acceptable carriers, additives and/or auxiliary substances are fillers, solvents, diluents, colorings and/or binders. These substances are known to the person skilled in the art (see H. P. Fiedler, Lexikon der Hilfsstoffe fur Pharmazie, Kosmetik and angrenzende Gebiete, Editio Cantor Aulendoff).

The pharmaceutical composition according to the invention contains the compound according to the invention in an amount of preferably from 0.001 to 99 wt. %, more preferably from 0.1 to 90 wt. %, yet more preferably from 0.5 to 80 wt. %, most preferably from 1.0 to 70 wt. % and in particular from 2.5 to 60 wt. %, based on the total weight of the pharmaceutical composition.

The pharmaceutical composition according to the invention is preferably for systemic, topical or local administration, preferably for oral administration.

Another aspect of the invention relates to a pharmaceutical dosage form which contains the pharmaceutical composition according to the invention.

In one preferred embodiment, the pharmaceutical dosage form according to the invention is produced for administration twice daily, for administration once daily or for administration less frequently than once daily. Administration is preferably systemic, in particular oral.

The pharmaceutical dosage form according to the invention can be administered, for example, as a liquid dosage form in the form of injection solutions, drops or juices, or as a semi-solid dosage form in the form of granules, tablets, pellets, patches, capsules, plasters/spray-on plasters or aerosols. The choice of auxiliary substances etc. and the amounts thereof to be used depend on whether the form of administration is to be administered orally, perorally, parenterally, intravenously, intraperitoneally, intradermally, intramuscularly, intranasally, buccally, rectally or locally, for example to the skin, the mucosa or into the eyes.

Pharmaceutical dosage forms in the form of tablets, dragées, capsules, granules, drops, juices and syrups are suitable for oral administration, and solutions, suspensions, readily reconstitutable dry preparations and also sprays are suitable for parenteral, topical and inhalatory administration. Compounds according to the invention in a depot, in dissolved form or in a plaster, optionally with the addition of agents promoting penetration through the skin, are suitable percutaneous administration preparations.

The amount of the compounds according to the invention to be administered to the patient varies in dependence on the weight of the patient, on the type of administration, on the indication and on the severity of the disease. Usually, from 0.00005 mg/kg to 50 mg/kg, preferably from 0.001 mg/kg to 10 mg/kg, of at least one compound according to the invention is administered.

Another aspect of the invention relates to a process for the preparation of the compounds according to the invention. Suitable processes for the synthesis of the compounds according to the invention are known in principle to the person skilled in the art.

Preferred synthesis routes are described below:

The compounds according to the invention can be obtained via different synthesis routes. Depending on the synthesis route, different intermediates are prepared and subsequently further reacted.

According to a first process, compounds of Formula (I) may be prepared from the compounds of Formulae (II) or (VIII) or (XI) and (TTT), as illustrated by Scheme 1.

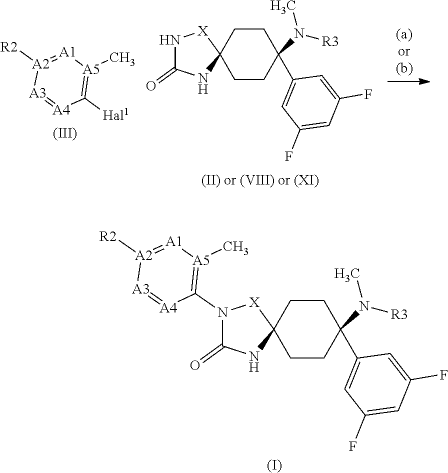

X represents CR4R5, i.e. either CH₂ or C ═O.

Hal¹ is a halogen, preferably Cl or Br or I.

The compound of Formula (I) may be prepared according to process step (a), an Ullmann-type copper-catalyzed arylation cross coupling reaction. Typical conditions comprise, reaction of the amine of Formula (II) or (VIII) or (XI) with the halide of Formula (III) in the presence of a suitable copper (II) catalyst, a suitable inorganic base and a suitable ligand in a suitable solvent and an appropriate temperature. Preferred conditions comprise, reaction of compounds of Formulae (II) or (VIII) or (XI) and (III) in the presence of CuI or Cu₂O, DMEDA and K₃PO₄ or K₂CO₃ in a suitable solvent such as dioxane or DMA at between 90° C. and 180° C. in a sealed tube. Alternatively, the compound of Formula (I) may be prepared according to process step (b), a Buchwald-Hartwig cross coupling reaction. Typical conditions comprise, reaction of the amine of Formula (II) or (VIII) or (XI) with the halide of Formula (III) in the presence of a suitable inorganic base, a suitable palladium catalyst in a suitable solvent at elevated temperature. Preferred conditions comprise, reaction of the compounds of Formulae (II) or (VIII) or (XI) and (III) in the presence of Xantphos in combination with Pd₂(dba)₃ in the presence of a suitable base such as Cs₂CO₃ in a suitable solvent such as dioxane at between 90° C. and 130° C. either under reflux or in a sealed vessel.

According to a second process compounds of Formula (II) may be prepared from compounds of Formulae (IV), (V), (VI), (VII) and (VIII) as illustrated by Scheme 2.

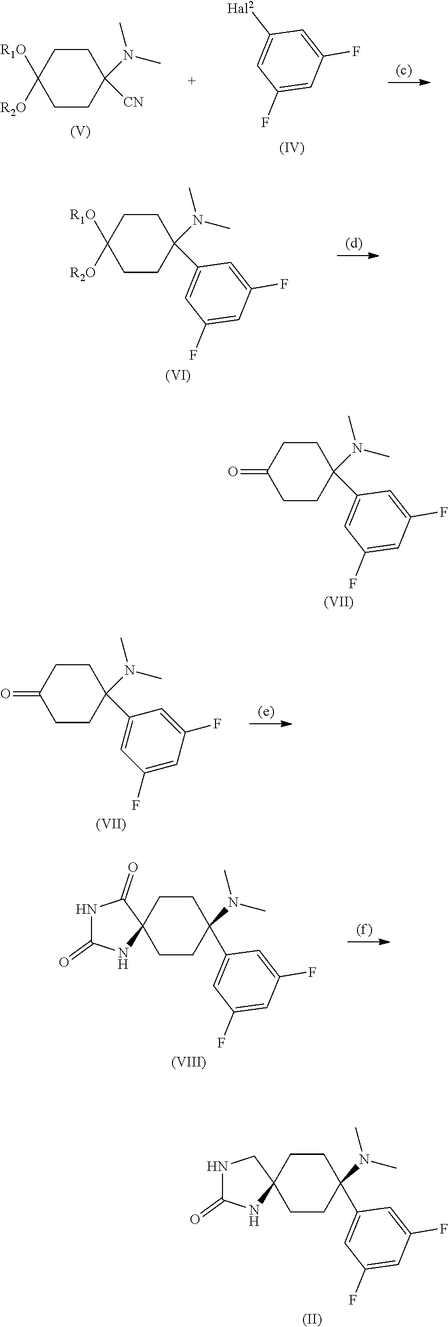

Hal² is a halogen, preferably Br

OR₁ and OR₂ are together a carbonyl protecting group, preferably 1,3-dioxolane

The compound of Formula (VI) may be prepared from the compounds of Formulae (IV) and (V) by process step (c) a organometallic displacement reaction. Typical conditions comprise the reaction of a halide of Formula (IV) with a suitable organometallic base such as a Grignard reagent, preferably iPrMgBr in a suitable aprotic solvent, preferably THF at a temperature between 15° C. and 50° C. followed by the addition of a compound of Formula (V) in an aprotic solvent such as THF.

The compound of Formula (VII) may be prepared from the compound of Formula (VI) according to a process step (d) a ketal deprotection reaction. Typical conditions comprise the reaction of a ketal of Formula (VI) with an appropriate acid, such as aqueous sulphuric acid at a suitable temperature such as rt.

The compound of Formula (VIII) may be prepared from the compound of Formula (VII) according to a process step (c) a Bucherer-Bergs reaction. Typical conditions comprise the reaction between a ketone of Formula (VII), potassium cyanide and ammonium carbonate in aqueous methanol at a suitable temperature of rt to 70° C., preferably at 70° C.

The compound of Formula (II) may be prepared from the compound of Formula (VIII) according to a process step (f) a reduction reaction. Typical conditions comprise the reaction of a compound of Formula (VIII) with a suitable reducing agent, preferably BH₃·DMS in a suitable aprotic solvent, preferably THF at a temperature of 0° C. to 65° C.

According to a third process compounds of Formula (XI) may be prepared from compounds of Formulae (II), (IX) and (X) as illustrated by Scheme 2.

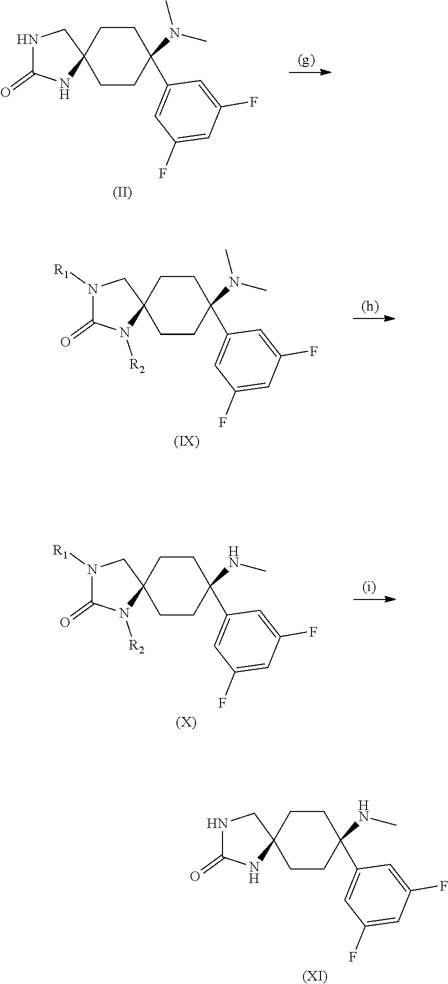

R₁ and R₂ are protecting groups, preferably R₁═R₂, preferably p-methoxy benzyl.

The compound of Formula (IX) may be prepared from the compounds of Formulae (II) by process step (g) an alkylation reaction. Typical conditions comprise the reaction of a urea of Formula (II) with a suitable alkylating reagent, preferably PMBCl and a suitable base, preferably t-BuOK in a suitable aprotic solvent, preferably DMSO at a temperature between 15° C. and 50° C.

The compound of Formula (X) may be prepared from the compound of Formula (IX) according to a process step (h) a demethylation reaction. Typical conditions comprise the reaction of an amine of Formula (IX) with an appropriate demethylation reagent, such as N-Iodosuccinimide in a suitable solvent, such as MeCN at a suitable temperature, such as rt.

The compound of Formula (XI) may be prepared from the compound of Formula (X) according to a process step (i) a deprotection reaction. Typical conditions comprise the reaction of a urea of Formula (X) with an appropriate acid, such as TFA at a suitable temperature such as 90° C.

The compounds of Formulae (III), (IV) and (V) are either commercially available or may be prepared by analogy to methods known in the literature, or the methods described in the Experimental section below.

Compounds of Formula (I) and (III) may be converted to alternative compounds of Formula (I) and (III) by standard chemical transformations, known to those skilled in the art. Examples of these transformations include, but are not limited to alkylation of a heteroatom, such as N or O, halogen interconversions, fluorination, nucleophilic reactions of ketones.

It will also be appreciated by those skilled in the art that it may be necessary to utilize a suitable protecting group strategy for the preparation of compounds of Formula (I) and (III).

It will be further appreciated that it may be necessary or desirable to carry out the transformations in a different order from that described in the schemes, or to modify one or more of the transformations, to provide the desired compound of the invention.

### EXAMPLES

“RT” means room temperature (23±7° C.), “M” are indications of concentration in mol/l, “aq.” means
aqueous, “sat.” means saturated, “sol.” means solution, “conc.” means concentrated.

Further abbreviations: Aq. means aqueous; BH₃·DMS means borane dimethyl sulfide complex; br means broad; ° C. means degrees Celsius; CDCl₃ means deutero-chloroform; Cs₂CO₃ means cesium carbonate; d means chemical shift; d means doublet; dd means double of doublets; DCM means dichloromethane; DABCO means 1,4-diazabicyclo[2.2.2]octane; DAST means diethylaminosulfur trifluoride; DMEDA means 1,2-dimethylethylenediamine; DMF means N,N-dimethylformamide; DMF-DMA means dimethylformamide dimethylacetal; DMSO means dimethylsulfoxide; DMSO-d₆ means hexadeuterodimethyl sulfoxide; Et means ethyl; EtOH means ethanol; EtOAc means ethyl acetate; Eq. means equivalent; g means gram; H₂SO₄ means sulfuric acid; HCl means hydrochloric acid; HCO₂H means formic acid; Hex means hexanes; H NMR means proton nuclear magnetic resonance; H₂O means water; HPLC means high pressure liquid chromatography; h means hour; IPA means 2-propanol; iPrMgBr means isopropyl magnesium bromide; KCN means potassium cyanide; K₂CO₃ means potassium carbonate; K₃PO₄ means potassium phosphate tribasic; L means liter; LCMS means liquid chromatography mass spectrometry; m means multiplet; M means molar; Me means methyl; MeCN means acetonitrile; MeI means iodomethane; MeOH means methanol; MeOH-d₄ means deutero-methanol; Me₃SiOK means potassium trimethylsilanolate; mg means milligram; MgSO₄ means magnesium sulfate; MHz means mega Hertz; min means minutes; mL means milliliters; mmol means millimole; MS m/z means mass spectrum peak; MTBE means tert-butyl methyl ether; N₂ means nitrogen; Na₂CO₃ means sodium carbonate; NaH means sodium hydride; NaHCO₃ means sodium bicarbonate; NaOH means sodium hydroxide; Na₂SO₄ means sodium sulfate; NH₄Cl means ammonium chloride; NH₄OH is ammonium hydroxide; (NH₄)₂CO₃ means ammonium carbonate; NIS means N-iodosuccinimide; PE means petroleum ether; q means quartet; rt means room temperature (23° C.); PMBCl means 4-methoxybenzylchloride; RT means retention time; s means singlet; sat. means saturated; soln. means solution; t means triplet; TEA means triethylamine; TFA means trifluoroacetic acid; THF means tetrahydrofuran; TLC means thin layer chromatography; μL means micro liters; gmol means micromole; Xanthphos means 4,5-bis(diphenylphosphino)-9,9-dimethylxanthene; XeF₂ means xenon difluoride.

The yields of the compounds prepared were not optimized. All temperatures are uncorrected. All starting materials, which are not explicitly described, were either commercially available (the details of suppliers such as for example Acros, Aldrich, Bachem, Butt park, Enamine, Fluka, Lancaster, Maybridge, Merck, Sigma, TCI, Oakwood, etc. can be found in the Symyx® Available Chemicals Database of MDL, San Ramon, US or the SciFinder® Database of the ACS, Washington DC, US, respectively, for example) or the synthesis thereof has already been described precisely in the specialist literature (experimental guidelines can be found in the Reaxys® Database of Elsevier, Amsterdam, NL or the SciFinder® Database of the ACS, Washington DC, US, respectively, for example) or can be prepared using the conventional methods known to the person skilled in the art.

The mixing ratios of solvents or eluents for chromatography are specified in v/v.

All the intermediate products and exemplary compounds were analytically characterized by mass spectrometry. In addition 1H-NMR and 13C spectroscopy was carried out for all the exemplary compounds and selected intermediate products.

As far as stereochemistry and its nomenclature is concerned, “cis” refers to the relative configuration of compounds described herein, in which both nitrogen atoms are drawn on the same face of the cyclohexane ring as described in the following exemplary structure. Two depictions are possible:

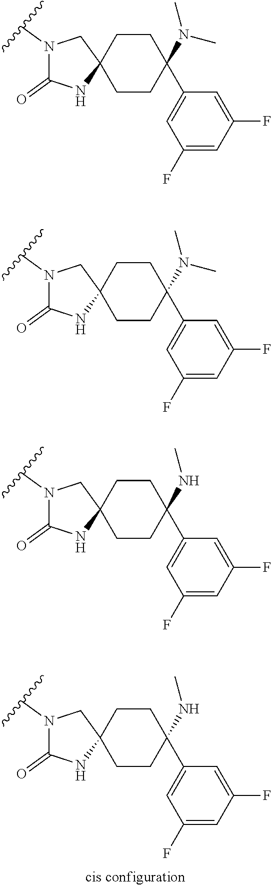

### Synthesis of Intermediates

### Synthesis of INT_001: 8-(3,5-difluorophenyl)-N,N-dimethyl-1,4-dioxaspiro[4.5]decan-8-amine

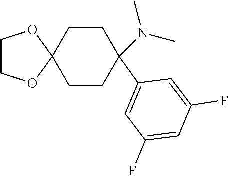 [SMILES: CN(C)C1(c2cc(F)cc(F)c2)CCC2(CC1)OCCO2]

A solution of 1-bromo-3,5-difluorobenzene (321 g, 1.66 mol) in THF (6 L) was added dropwise slowly to a solution of iPrMgBr (832 mL, 2M) at 15° C. and the mixture warmed slowly to 50° C. The solution was re-cooled to 15° C. and stirred for 2 h before being added dropwise over 1 h to a solution of 8-(dimethylamino)-1,4-dioxaspiro[4.5]decane-8-carbonitrile (100 g, 476 mmol) in THF (600 mL) at 0° C. The resulting mixture was stirred at 15° C. for 12 h. The reaction mixture was added slowly to sat. NH₄Cl (1.70 L) and extracted with EtOAc (1 L×2). The combined organics were washed with brine (500 mL), dried (Na₂SO₄) and evaporated to dryness in vacuo to afford the title compound as an oil (157 g, crude). ¹H NMR (400 MHz, CDCl₃) δ: 6.84 (br d, 2H), 6.77-6.62 (m, 1H), 3.99-3.90 (m, 4H), 2.26-2.16 (m, 2H), 2.11-2.01 (m, 8H), 1.90-1.81 (m, 2H), 1.54-1.46 (m, 2H).

### Synthesis of INT_002: 4-(3,5-difluorophenyl)-4-(dimethylamino)cyclohexan-1-one

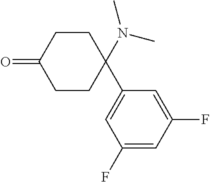 [SMILES: CN(C)C1(c2cc(F)cc(F)c2)CCC(=O)CC1]

Two reactions of equal scale were carried out in parallel. 8-(3,5-difluorophenyl)-N,N-dimethyl-1,4-dioxaspiro[4.5]decan-8-amine (INT_001, 275 g, 925 mmol) was added to H₂SO₄ (1.36 kg, 1.39 mol, 10% w/w aqueous solution) at 15° C. and the resulting mixture stirred at 15° C. for 16 h. The reaction mixture was extracted with DCM (1 L). The aqueous phase was adjusted to pH >11 by the addition of sat. NaOH and the solids collected by filtration. The filter cake was washed with H₂O (500 mL) and the solids dried under reduced pressure to give the title compound as a solid (190 g, crude). ¹H NMR (400 MHz, DMSO-d₆) δ: 6.83-6.59 (m, 3H), 2.65-2.44 (m, 4H), 2.21 (dt, 2H), 2.07 (s, 6H), 2.03-1.95 (m, 2H).

### Synthesis of INT_003: cis-8-(3,5-difluorophenyl)-8-(dimethylamino)-1,3-diazaspiro[4.5]-decane-2,4-dione

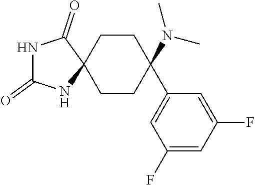 [SMILES: CN(C)[C@]1(c2cc(F)cc(F)c2)CC[C@@]2(CC1)NC(=O)NC2=O]

Four reactions were carried out in parallel. 4-(3,5-difluorophenyl)-4-(dimethylamino)cyclohexan-1-one (INT_002, 125 g, 494 mmol) was dissolved in H₂O (1.25 L) and MeOH (1.25 L) at 15° C. To this was added (NH₄)₂CO₃ (119 g, 1.23 mol) and KCN (32.4 g, 498 mmol) and the solution was stirred at 70° C. for 16 h. The four parallel reactions were combined and the solids collected by filtration. The filter cake was washed with H₂O (2 L) and MeOH (2 L) and the solids triturated with MeOH (500 mL) at 70° C. for 16 h. The solids were collected and dried under reduced pressure to give the title compound as a solid (570 g, crude) which was used without additional purification. ¹H NMR (400 MHz, DMSO-d₆) δ: 8.31-8.19 (m, 1H), 7.16-7.10 (m, 1H), 7.02 (br d, 2H), 3.45-3.20 (m, 1H), 2.46-2.37 (m, 2H), 2.08-1.84 (m, 8H), 1.55 (br d, 2H), 1.47-1.30 (m, 2H).

### Synthesis of INT_004: cis-8-(3,5-difluorophenyl)-8-(dimethylamino)-1,3-diazaspiro[4.5]decan-2-one

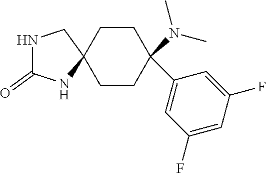 [SMILES: CN(C)[C@]1(c2cc(F)cc(F)c2)CC[C@]2(CC1)CNC(=O)N2]

Two reactions were carried out in parallel. BH₃·DMS (711 mL, 10 M) was added to a solution of cis-8-(3,5-difluorophenyl)-8-(dimethylamino)-1,3-diazaspiro[4.5]decane-2,4-dione (INT_003, 230 g, 711 mmol) in THF (1.3 L) at 0° C. and the resulting mixture stirred at 65° C. for 16 h. The reaction mixture was added very slowly to HCl (6M, 1 L) followed by MeOH (1 L) and H₂O (1 L) and allowed to stir for 10 min. The solids were removed by filtration and the filter cake washed with H₂O (500 mL) and MeOH (500 mL). The pH of the filtrate was adjusted to pH >13 by addition of sat. NaOH. The solids were collected by filtration and the filter cake washed with H₂O (2 L) and dried under reduce pressure to give a residue. The residue was added to HCl (500 mL, 6 M) and stirred for 10 min before re-collecting to the solids and washing with H₂O (100 mL). The solids were again added to H₂O (150 mL) and stirred for 10 mins, collected by filtration and washed with H₂O (100 mL). The process was repeated and the collected solids dried under reduced pressure. The solids were added to H₂O (500 mL), stirred for 2 mins and then the pH adjusted to pH >12 with sat. NaOH. Stirring was continued for 10 min and the solids collected by filtration, washed with H₂O (500 mL) and dried under reduced pressure to give the title compound as a solid (200 g, 647 mmol). ¹H NMR (400 MHz, DMSO-d₆) δ: 7.12 (t, 1H), 7.07-6.99 (m, 2H), 6.60 (br s, 1H), 6.02 (s, 1H), 3.01 (s, 2H), 2.24 (br s, 2H), 1.94 (s, 6H), 1.77 (br d, 4H), 1.44-1.27 (m, 2H)

### Synthesis of INT_005: 2-bromo-5-cyclopropoxy-3-methylpyridine

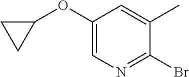

Twenty-four equivalent reactions were carried out in parallel. A mixture of 6-bromo-5-methylpyridin-3-ol (1.50 g, 7.98 mmol), Cs₂CO₃ (3.38 g, 10.4 mmol), KI (199 mg, 1.20 mmol), and bromocyclopropane (3.86 g, 31.9 mmol) in DMF (10 mL) was stirred at 140° C. for 2 h under microwave conditions. The Twenty-four reactions were combined and the solids removed by filtration and the filter cake washed with EtOAc (50 mL). The filtrate was extracted with EtOAc (80 mL×4) and the combined organics were evaporated to dryness in vacuo. The residue was purified by column chromatography (SiO₂, 25-100% EtOAc/PE) to afford the title compound as a solid (7.00 g, 15.7% yield). ¹H NMR (400 MHz, DMSO-d₆) δ: 8.03 (d, 1H), 7.53 (d, 1H), 3.95 (tt, 1H), 2.31 (s, 3H), 0.87-0.65 (m, 4H).

### Synthesis of INT_006: 5-bromo-2-methoxy-4-methylpyrimidine

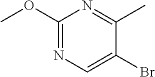

To a stirred solution of 5-bromo-2-chloro-4-methylpyrimidine (5 g, 24.1 mmol) in THF (120 mL) was added NaOMe (25% in MeOH; 6.05 mL, 26.49 mmol) at 0° C. and stirred at rt for 16 h. The reaction mixture was cooled to 0° C. and quenched with saturated aq. NH4Cl solution and extracted with EtOAc (200 mL). The combined organics were washed with water (100 mL), brine (100 mL), dried (Na₂SO₄) and evaporated to dryness in vacuo. The residue was purified by combi-flash column chromatography (0-5% EtOAc/Hexane) to afford the title compound as an oil (3.6 g, 73%). LCMS m/z=205 [M+H]⁺.

### Synthesis of INT_007: 2-(6-chloro-5-methylpyridin-3-yl)propan-2-ol

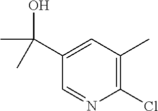

iPrMgCl·LiCl (1.30 M, 117 mL) was added dropwise to a mixture of molecular sieves (5 Å, 10 g) and 5-bromo-2-chloro-3-methylpyridine (30.0 g, 145 mmol) in THF (500 mL) at 0° C. and the mixture stirred for 1 h at 0° C. Acetone (12.7 g, 218 mmol) was added dropwise over 1 h to the reaction mixture at 0° C. and the mixture was stirred at 0° C. for 1 h. The reaction was quenched with NH₄Cl (500 mL) and extracted with EtOAc (3×500 mL). The combined organics were washed with brine, dried (Na₂SO₄) and evaporated to dryness in vacuo. The residue was purified by column chromatography (SiO₂, 50:1-0:1 PE/EtOAc) to afford the title compound as an oil (15 g, 53%). LCMS m/z=186 [M+H]⁺.

### Synthesis of INT_008: 2-bromo-5-(difluoromethoxy)-3-methylpyridine

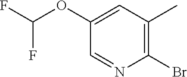

A mixture of 6-bromo-5-methylpyridin-3-ol (31.0 g, 165 mmol), sodium 2-chloro-2,2-difluoroacetate (126 g, 829 mmol), Cs₂CO₃ (39.2 g, 120 mmol) and K₂CO₃ (16.6 g, 120 mmol) in DMF (310 mL) was stirred at 100° C. for 16 h. The reaction mixture was filtered and the filter cake washed with EtOAc. The filtrate was extracted with EtOAc (250 mL×2) and water (300 mL). The combined organics were washed with brine (200 mL×2) and evaporated to dryness under reduced pressure. The residue was purified by column chromatography (SiO₂, 5:1 PE/Ethyl acetate) to afford the title compound as an oil (16.8 g, 42.8%). ¹H NMR (400 MHz, CDCl₃) δ: 8.11 (s, 1H), 7.40-7.34 (m, 1H), 6.77-6.35 (m, 1H), 2.42 (s, 3H).

### Synthesis of INT_009: 3-bromo-4-methyl-5-nitropyridin-2(1H)-one

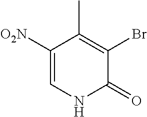

To a stirred solution of 4-methyl-5-nitro-1,2-dihydropyridin-2-one (6.0 g, 38.961 mmol) in AcOH (30 ml) was added bromine (2.4 ml, 46.7 mmol) at room temperature and stirred for 16 h at that temperature. The reaction mixture was poured into ice whereby the product was precipitated as a white solid. The product was collected by filtration and washed with H₂O before being to obtain the title compound as a solid (8 g, 88%). LCMS m/z=233 [M+H]⁺.

### Synthesis of INT_010: 3-bromo-2-chloro-4-methyl-5-nitropyridine

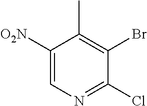

To a stirred solution of 3-bromo-4-methyl-5-nitro-1,2-dihydropyridin-2-one (INT_009, 5.0 g, 21.5 mmol) in MeCN were added DIPEA (3.7 ml, 21.5 mmol) and POCl₃ (20.0 ml, 214 mmol) at 0° C. The reaction mixture was stirred at 80° C. for 3 h. The reaction mixture was concentrated under reduced pressure, ice cold water (30 ml) was added and the resulting mixture was basified by addition of sat. aq. NaHCO₃ solution. This mixture was extracted with EtOAc (60 ml). The organic layer was dried over anhydrous Na₂SO₄ and concentrated in vacuo. The residue was purified by column chromatography to afford the title compound as a solid (4.3 g, 79%). LCMS m/z=251 [M+H]⁺.

### Synthesis of INT_011: (E)-2-(3-bromo-2-chloro-5-nitropyridin-4-yl)-N,N-dimethylethen-1-amine

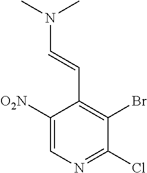 [SMILES: CN(C)/C=C/c1c([N+](=O)[O-])cnc(Cl)c1Br]

A solution of 3-bromo-2-chloro-4-methyl-5-nitropyridine (INT_010, 4.3 g, 17.1 mmol) in DMF-DMA (25 ml) was stirred at 50° C. for 16 h. The reaction mixture was concentrated under reduced pressure. The residue was purified by column chromatography (SiO₂, 20% EtOAc/hexanes) to afford the title compound as a solid (5.0 g, 95%). LCMS m/z=306 [M+H]⁺.

### Synthesis of INT_012: 4-bromo-5-chloro-1H-pyrrolo[2,3-c]pyridine

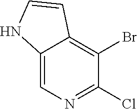 [SMILES: Clc1ncc2[nH]ccc2c1Br]

To a solution of [(E)-2-(3-bromo-2-chloro-5-nitropyridin-4-yl)ethenyl]dimethylamine (INT_011, 5 g, 16.3 mmol) in EtOH and H₂O were added Fe (4.6 g, 81.6 mmol) and NH₄Cl (8.7 g, 163.1 mmol) and the reaction mixture was stirred at 100° C. for 2 h. The reaction mixture was filtered, concentrated under reduced pressure and diluted with EtOAc (60 ml). This solution was washed with water (30 ml) and sat. NaCl solution (30 ml). The organic layer was dried over anhydrous Na₂SO₄ and concentrated in vacuo. The residue was purified by column chromatography (SiO₂, 20% EtOAc/hexanes) to afford the title compound as a solid (1.6 g, 42%). LCMS m/z=231 [M+H]⁺.

### Synthesis of INT_013: 5-chloro-4-methyl-1H-pyrrolo[2,3-c]pyridine

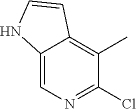

To a stirred solution of 4-bromo-5-chloro-1H-pyrrolo[2,3-c]pyridine (INT_012, 1.0 g, 4.32 mmol) in 1,4-dioxane and H₂O were added trimethylboroxine (0.73 ml, 5.2), K₂CO₃ (1.791 g, 12.959 mmol, 3 equiv.) and Pd(dppf)Cl₂ (0.32 g, 0.43 mmol). The reaction mixture was stirred for 16 h at 90° C. before being filtered. The filtrate was concentrated under reduced pressure and the residue was purified by column chromatography (SiO₂, 35% EtOAc/hexanes) to afford the title compound as a solid (0.3 g, 42%). LCMS m/z=167 [M+H]⁺.

### Synthesis of INT_014: 5-chloro-4-methyl-1-((2-(trimethylsilyl)ethoxy)methyl)-1H-pyrrolo[2,3-c]pyridine

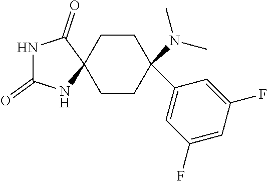 [SMILES: CN(C)[C@]1(c2cc(F)cc(F)c2)CC[C@@]2(CC1)NC(=O)NC2=O]

To a stirred solution of 5-chloro-4-methyl-1H-pyrrolo[2,3-c]pyridine (INT_013, 0.15 g, 0.9 mmol) in DMF (5 ml) was added NaH (60% in mineral oil, 0.07 g, 1.8 mmol) at 0° C. and stirred for 20 min. SEM-Cl (0.24 ml, 1.35 mmol) was added to the reaction mixture and stirred at room temperature for 1 h. The reaction mixture was quenched by addition of aq. NH₄Cl solution, extracted with EtOAc (30 mL) and washed with H₂O (10 mL) and sat. NaCl solution (10 mL). The combined organic layers were dried over anhydrous Na₂SO₄ and concentrated under reduced pressure. The residue was purified by column chromatography (SiO₂, 10% EtOAc/hexanes) to afford the title compound (0.2 g, 74%). LCMS m/z=297 [M+H]⁺.

### Synthesis of INT_015: cis-8-(3,5-difluorophenyl)-8-(dimethylamino)-3-(4-methyl-1-((2-(trimethylsilyl)ethoxy)methyl)-1H-pyrrolo[2,3-c]pyridin-5-yl)-1,3-diazaspiro[4.5]decan-2-one

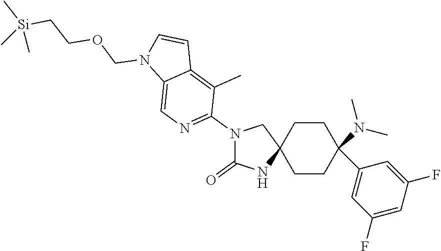 [SMILES: Cc1c(N2C[C@]3(CC[C@](c4cc(F)cc(F)c4)(N(C)C)CC3)NC2=O)ncc2c1ccn2COCC[Si](C)(C)C]

To a stirred solution of cis-8-(3,5-difluorophenyl)-8-(dimethylamino)-1,3-diazaspiro[4.5]decan-2-one (INT_004, 0.15 g, 0.49 mmol) in 1,4-dioxane (5 mL) was added 5-chloro-4-methyl-1-{[2-(trimethylsilyl)ethoxy]methyl}-1H-pyrrolo[2,3-c]pyridine (INT 014, 0.173 g, 0.58 mmol) and K₂CO₃ (0.2 g, 1.5 mmol). The mixture was degassed for 5 minutes by purging with argon. Then trans-N,N′-dimethylcyclohexane-1,2-diamine (0.015 ml, 0.096 mmol) and CuI (0.009 g, 0.048 mmol) were added and the resulting mixture was stirred at 130° C. for 16 h. The reaction mixture was cooled to room temperature and filtered through a pad of celite. The filtrate was concentrated under reduced pressure and the residue was diluted with 10% MeOH in DCM (50 mL) and then washed with water (20 mL) and sat. NaCl solution (20 mL). The organic layer was dried over anhydrous Na₂SO₄ and concentrated under reduced pressure. The residue was purified by column chromatography (SiO₂, 2-3% MeOH/DCM) to afford the title compound (0.2 g, 72%). LCMS m/z=570 [M+H]⁺.

### Synthesis of INT_016: cis-(3,5-difluorophenyl)-8-(dimethylamino)-1,3-bis(4-methoxybenzyl)-1,3-diazaspiro[4.5]decan-2-one

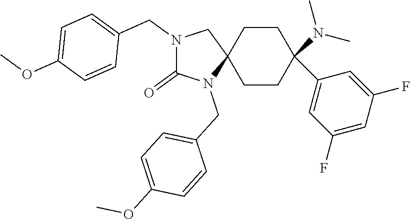 [SMILES: COc1ccc(CN2C[C@]3(CC[C@](c4cc(F)cc(F)c4)(N(C)C)CC3)N(Cc3ccc(OC)cc3)C2=O)cc1]

To a solution of cis-8-(3,5-difluorophenyl)-8-(dimethylamino)-1,3-diazaspiro[4.5]decan-2-one (INT_004, 10.0 g, 32.2 mmol) in DMSO (100 mL) was added t-BuOK (10.8 g, 96.7 mmol) and PMBCl (13.0 mL, 19.7 mmol) at room temperature and the mixture was stirred for 2 h at that temperature The reaction was quenched by addition of H₂O (150 mL) and extracted with EtOAc (2×150 mL). The combined organic layers were washed with sat. NaCl solution (100 mL), dried over anhydrous Na₂SO₄ and concentrated in vacuo. The residue was purified by column chromatography (SiO₂, 20-30% EtOAc/PE) to afford the title compound (14.0 g, 79%) as a solid. LCMS m/z=550.9 [M+H]⁺.

### Synthesis of INT_017: cis-8-(3,5-difluorophenyl)-1,3-bis(4-methoxybenzyl)-8-(methylamino)-1,3-diazaspiro[4.5]decan-2-one

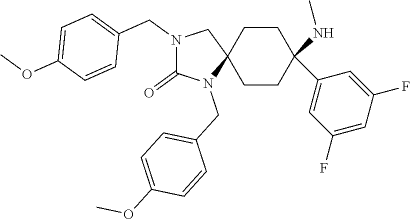 [SMILES: CN[C@]1(c2cc(F)cc(F)c2)CC[C@]2(CC1)CN(Cc1ccc(OC)cc1)C(=O)N2Cc1ccc(OC)cc1]

To a solution of cis-8-(3,5-difluorophenyl)-8-(dimethylamino)-1,3-bis(4-methoxybenzyl)-1,3-diazaspiro[4.5]decan-2-one (INT_016, 17.1 g, 31.1 mmol) in MeCN (340 mL) was added NIS (10.5 g, 46.6 mmol) and stirred at room temperature for 1 h. The reaction was quenched by addition of diluted aqueous Na₂S₂O₃ solution (100 mL) and extracted with EtOAc (2×150 mL). The combined organic layer was washed with sat. NaCl solution (100 mL), dried over anhydrous Na₂SO₄ and concentrated in vacuo. The residue was purified by column chromatography (SiO₂, 30-40% EtOAc/PE) to afford the title compound (13.1 g, 79%) as a solid. LCMS m/z=536.9 [M+H]⁺.

### Synthesis of INT_018: cis-8-(3,5-difluorophenyl)-8-(methylamino)-1,3-diazaspiro[4.5]decan-2-one

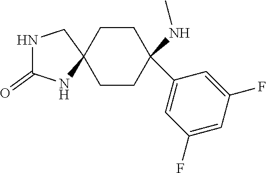 [SMILES: CN[C@]1(c2cc(F)cc(F)c2)CC[C@]2(CC1)CNC(=O)N2]

Cis-8-(3,5-difluorophenyl)-1,3-bis(4-methoxybenzyl)-8-(methylamino)-1,3-diazaspiro[4.5]decan-2-one (INT_017, 13.0 g, 24.3 mmol) was dissolved in TFA (130 mL) and stirred at 90° C. for 18 h. The reaction mixture was concentrated under reduced pressure and washed with EtOAc (100 mL), basified with aqueous NaHCO₃ solution (500 mL) and extracted with 10% MeOH/DCM (2×500 mL). The combined organic layers were washed with sat. NaCl solution (200 mL), dried over anhydrous Na₂SO₄ and concentrated in vacuo to afford the title compound (6.0 g, 84%) as a solid. LCMS m/z=296.2 [M+H]⁺.

### Synthesis of INT_019: 5-bromo-2-cyclopropoxy-4-methylpyrimidine

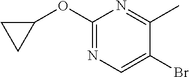

Cyclopropyl alcohol (15.1 g, 260 mmol) in THF (40 mL) was added dropwise over 30 min to a suspension of NaH (10.4 g, 260 mmol) in THF (800 mL) at 0° C. and the mixture was stirred at 0° C. for 30 min. To this was added dropwise, over 30 min, a solution of 5-bromo-2-chloro-4-methylpyrimidine (45.0 g, 217 mmol) in THF (80 mL) at 0° C. and the resulting mixture stirred at 25° C. for 16 h. The reaction was quenched by the addition of sat. aq. NH₄Cl (1 L) at 10° C. and then extracted with EtOAc (3×400 mL). The combined organics were dried and evaporated to dryness under reduced pressure. The residue was purified by column chromatography (SiO₂, 16-100% EtOAc/PE) to give the title compound as an oil (19.0 g, 38%). ¹H NMR (400 MHz, DMSO-d₆) δ: 8.67 (s, 1H), 4.34-4.21 (m, 1H), 2.49 (s, 3H), 0.81-0.75 (m, 2H), 0.74-0.68 (m, 2H).

### Synthesis of INT_020: 4-bromo-1,3-dimethylpyridin-2(1H)-one

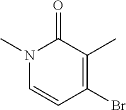

MeI (16.6 mL, 266 mmol) was added to a solution of 4-bromo-3-methylpyridin-2(1H)-one (1) (5.0 g, 26.6 mmol) and t-BuOK (5.96 g, 53.2 mmol) in 1,4-dioxane (250 mL) at rt under argon and heated at 80° C. for 18 h. The reaction mixture was cooled to rt, filtered through celite pad and washed with DCM (2×20 mL). The filtrate was evaporated to dryness in vacuo and the residue purified by flash chromatography (70-80% EtOAc/PE) to afford the title compound as a solid (5.3 g, 98%). LCMS m/z=202 [M+H]⁺.

### Synthesis of INT_021: 2-(5-chloro-6-methylpyrazin-2-yl)propan-2-ol

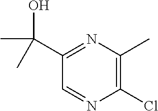

To a stirred solution of methyl 5-chloro-6-methylpyrazine-2-carboxylate (3.5 g, 18.75 mmol) in THF (100 mL) was added MeMgBr (3M in Et₂O; 19 ml, 56.25 mmol) dropwise at 0° C. and the mixture was stirred at 0° C. for 2 h. The reaction was quenched with sat. aq. NH₄Cl solution (50 mL) and extracted with EtOAc (2×80 mL). The combined organics were dried (Na₂SO₄) and evaporated to dryness in vacuo. The residue was purified by column chromatography (SiO₂, 0-20% EtOAc/Hex) to afford the title compound as an oil (2.2 g, 63%). LCMS m/z=187 [M+H]⁺.

### Synthesis of INT_022: 5-bromo-2-iodo-4-methylpyrimidine

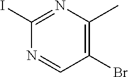

To a solution of 5-bromo-2-chloro-4-methylpyrimidine (1.0 g, 4.83 mmol) in DCM (10 mL) was added HI in water (0.92 mL, 20.77 mmol) at rt and the mixture stirred at rt for 16 h. The reaction mixture was quenched with ice-water (50 mL) and extracted with EtOAc (2×100 mL). The combined organics were washed with water (50 mL), brine solution (50 mL), dried (Na₂SO₄) and evaporated to dryness in vacuo. The reside was purified by column chromatography (SiO₂, 0-10% EtOAc/PE) to afford the title compound as a solid (900 mg, 63%). LCMS m/z=299 [M+H]⁺.

### Synthesis of INT_023: 3-(5-bromo-4-methylpyrimidin-2-yl)oxetan-3-ol

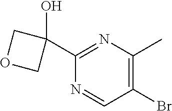

To a solution of 5-bromo-2-iodo-4-methylpyrimidine (INT_022, 0.900 g, 3.03 mmol) in toluene (25 mL) was added n-butyl lithium (1.98 mL, 3.18 mmol, 1.6M in n-hexane) at −78° C. and the reaction mixture stirred at −78° C. for 30 min. Oxetan-3-one (0.213 mL, 3.64 mmol) was added to the reaction mixture at −78° C. and the reaction mixture stirred at −78° C. for 30 min. The reaction was quenched with saturated sodium sulphate solution and extracted with EtOAc (2×50 mL). The combined organics were washed with H₂O (50 mL), brine (50 mL), dried (Na₂SO₄) and evaporated to dryness in vacuo. The residue was purified by column chromatography (SiO₂, 0-10% EtOAc/PE) to afford the title compound as an oil (300 mg, 40%). LCMS m/z=245 [M+H]⁺.

### Synthesis of INT_024: 5-bromo-2-(3-fluorooxetan-3-yl)-4-methylpyrimidine

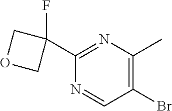

To a solution of 3-(5-bromo-4-methylpyrimidin-2-yl)oxetan-3-ol (INT_023, 400 mg, 1.65 mmol) in DCM (10 mL) was added DAST (0.4 mL, 3.29 mmol) at 0° C. and the mixture stirred at rt for 1 h. The reaction was quenched with ice-water (20 mL) and extracted with DCM (2×500 mL). The combined organics were washed with saturated sodium bicarbonate solution (50 mL), dried (Na₂SO₄) evaporated to dryness under reduced pressure. The residue was purified by column chromatography (SiO₂, 0-10% EtOAc/PE) to afford the title compound as an oil (240 mg, 59%). ¹H NMR (400 MHz, CDCl₃) δ: 8.79 (s, 1H), 5.20-5.00 (m, 4H), 2.70 (s, 3H).

### Synthesis of INT_025: 5-bromo-4-methylpyrimidine-2-carbonitrile

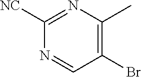

DABCO (12.24 g, 109 mmol) followed by NaCN (4.28 g, 87.4 mmol) were added to a solution of 5-bromo-2-chloro-4-methylpyrimidine (15 g, 72.8 mmol) in DMSO (150 mL) and H₂O (150 mL) and the mixture stirred at rt for 16 h. The reaction mixture was extracted with EtOAc (2×250 mL) and the combined organics were washed with H₂O (2×250 mL), brine (2×250 mL), dried (Na₂SO₄) and evaporated to dryness in vacuo. The residue was purified by Combi-Flash chromatography (5-10% EtOAc/Hex) to afford the title compound as a solid (11.5 g, 80%). LCMS m/z=198 [M+H]⁺.

### Synthesis of INT_026: methyl 5-bromo-4-methylpyrimidine-2-carboxylate

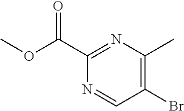

To a stirred solution of 5-bromo-4-methylpyrimidine-2-carbonitrile (INT_025, 1 g, 5.05 mmol) in MeOH (12 mL) was added conc. HCl (12 mL) and the reaction mixture stirred at 80° C. for 8 h. The reaction mixture was poured into cold sat. NaHCO₃ solution and extracted with EtOAc (2×50 mL). The combined organics were washed with H₂O (2×25 mL) and brine (2×25 mL), dried (Na₂SO₄) and concentrated under reduced pressure. The residue was purified by Combi-Flash column chromatography (10-50% EtOAc/Hex) to afford the title compound as a solid (434 mg, 37%). LCMS m/z=233 [M+H]⁺.

### Synthesis of INT_027: 2-(5-bromo-4-methylpyrimidin-2-yl)propan-2-ol

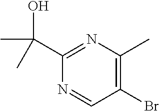

The title compound was prepared as a solid (0.25 g, 62%) from methyl 5-bromo-4-methylpyrimidine-2-carboxylate (INT_026) using an analogous method to that described for INT_20. LCMS m/z=231 [M+H]⁺.

### Synthesis of INT_028: 6-chloro-3-(1,1-difluoroethoxy)-2-methoxypyridine

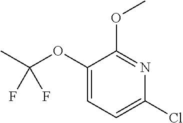

To a solution of 6-chloro-2-methoxynicotinaldehyde (40.0 g, 233 mmol) in THF (800 mL) was added MeMgBr (3.0 M, 117 mL) at 0° C. and the mixture was stirred for 30 min at that temperature. The reaction was quenched by addition of sat. aqueous NH₄Cl solution and the mixture was extracted with EtOAc (2×800 ml). The combined organic layers were dried over anhydrous Na₂SO₄ and evaporated to dryness under reduced pressure to yield the corresponding crude alcohol (48.0 g).

The residue (25.0 g, 133 mmol) was dissolved in MeCN (375 mL) and cooled to 0° C. under nitrogen atmosphere. Subsequently, an ice cold solution of NaIO₄ (57.0 g, 266 mmol) and RuCl₃ (691 mg, 3.33 mmol) in H₂O (300 mL) was added dropwise to the organic solution and the mixture was stirred for 1 h at room temperature. H₂O (300 mL) was added and the mixture was extracted with EtOAc (2×300 mL). The combined organic layers were dried over anhydrous Na₂SO₄ and concentrated in vacuo to afford the corresponding ketone as a solid (18.5 g, crude).

To a solution of the crude residue (2.0 g, 10.8 mmol) in DCM (60 mL) was added HF pyridine (12.8 g, 129 mmol) at room temperature and the mixture was stirred for 1 h. XeF₂ (5.47 g, 32.3 mmol) was added and the mixture was stirred for 12 h at room temperature. The reaction was quenched by addition of water (15 mL) and the pH was adjusted to 8-9 by addition of aqueous sat. Na₂CO₃ solution. The mixture was extracted with EtOAc (3×15 mL) and the combined organic layers were dried over anhydrous Na₂SO₄ and concentrated under reduced pressure. The residue was purified by column chromatography (SiO₂, 1-100% EtOAc/PE) to afford the title compound as an oil (1.17 g, 48% yield). ¹H NMR (400 MHz, CDCl₃) δ: 1.92-2.00 (m, 3H) 3.98-4.01 (m, 3H) 6.88 (d, 1H) 7.46 (d, 1H).

### Synthesis of INT_029: 6-chloro-3-(1,1-difluoroethoxy)-1-methylpyridin-2(1H)-one

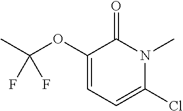

To a solution of 6-chloro-3-(1,1-difluoroethoxy)-2-methoxypyridine (INT 028, 4.5 g, 20.1 mmol) in MeCN (200 mL) was added NaI (15.1 g, 101 mmol) and TMSCl (10.9 g, 101 mmol) at 0° C. under nitrogen atmosphere. The reaction mixture was heated to 70° C., stirred for 2 h at that temperature and subsequently filtered through a pad of diatomite. The pad was washed with 1:10 MeOH/DCM (500 mL) and the combined organic layers were concentrated in vacuo. The residue was purified by column chromatography (SiO₂, 1-100% EtOAc/PE) to yield the 0-demethylated intermediate as a solid (4.12 g).

To a solution of above intermediate (2.15 g, 10.3 mmol) in acetone (10 mL) was added CH₃I (1.53 g, 10.8 mmol) and K₂CO₃ (2.84 g, 20.5 mmol) and the mixture was degassed by purging with N₂. The mixture was stirred for 16 h at room temperature before being quenched by addition with H₂O (10.0 mL). The mixture was extracted with DCM (10×10 mL) and the combined organic layers were dried over Na₂SO₄ and concentrated in vacuo. The residue was purified by column chromatography (SiO₂, 1-100% EtOAc/PE) to yield the title compound as an oil (1.49 g, 65%). ¹H NMR (400 MHz, CDCl₃) δ: 1.98 (t, 3H) 3.71-3.73 (m, 3H) 6.25 (d, 1H) 7.23 (d, 1H).

### Synthesis of INT_030: 1-(6-chloro-5-methylpyridin-3-yl)ethan-1-ol

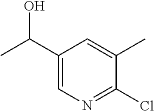

To a solution of 6-chloro-5-methylnicotinaldehyde (1.0 g, 6.426 mmol, 1.0 equiv.) in tetrahydrofuran (20 mL) was added MeMgBr (3.0M in Et₂O, 6.4 mL, 1.28 mmol) at 0° C. and the reaction mixture was stirred at room temperature for 2 h. The reaction mixture was quenched by addition of sat. aq. NH₄Cl solution (10 mL), diluted with ice-water (100 mL) and extracted with EtOAc (2×250 mL). The combined organic layers were washed with H₂O (50 mL), sat. NaCl solution (50 mL), dried over anhydrous Na₂SO₄ and the solvent was removed in vacuo. The crude product was purified by column chromatography (SiO₂, 10% EtOAc/PE) to afford the title compound as a solid (0.74 g, 67%). LCMS m/z=172 [M+H]⁺.

### Synthesis of INT_031: 2-(6-chloro-5-methylpyridin-3-yl)-1,1,1-trifluoropropan-2-ol

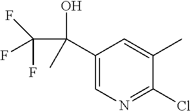

To a solution of 1-(6-chloro-5-methylpyridin-3-yl)ethan-1-ol (INT_030, 0.740 g, 4.327 mmol) in DCM (10 mL) was added PCC (1.86 g, 8.654 mmol) at room temperature and the reaction mixture was stirred at that temperature for 2 h. The reaction mixture was filtered through a pad of celite and the filter cake was washed with EtOAc (200 mL). The filtrate was concentrated in vacuo. The crude product was purified by column chromatography (SiO₂ 16% EtOAc/PE) to afford the corresponding ketone as a liquid (0.60 g).

To a solution of above ketone (0.50 g, 2.958 mmol) in THF (25 mL) were added trimethyl(trifluoromethyl)silane (0.87 mL, 5.917 mmol) and tetra-n-butyl ammonium fluoride (LOM in THF, 0.6 mL, 0.59 mmol) at 0° C. and the reaction mixture was stirred at room temperature for 1 h. The reaction mixture was diluted with ice-water (100 mL) and extracted with EtOAc (2×250 mL). The combined organic layer was washed with H₂O (50 mL), sat. NaCl solution (50 mL), dried over anhydrous Na₂SO₄ and the solvent was removed in vacuo. The residue was purified by column chromatography (SiO₂, 8% EtOAc/PE) to afford the title compound as a solid (0.54 g, 76%). LCMS m/z=240 [M+H]⁺.

### Synthesis of INT_032: 2-chloro-5-(2,2-difluoroethoxy)-3-methylpyridine

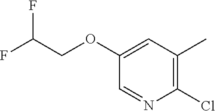

To a stirred solution of 6-chloro-5-methylpyridin-3-ol (0.3 g, 2.1 mmol) in DMF (5.0 mL) was added Cs₂CO₃ (1.0 g, 3.1 mmol) and 2,2-difluoroethyl trifluoromethanesulfonate (0.49 mg, 2.3 mmol) at room temperature. The reaction mixture was stirred at the same temperature for 2 h. The reaction mixture was diluted with water (50 mL) and extracted with EtOAc (2×50 mL). The combined organic layers were washed with sat. NaCl solution (50 mL), dried over anhydrous Na₂SO₄ and concentrated in vacuo to afford the title compound (0.4 g, 92%) as a solid which was used without further purification.

### Synthesis of INT_033: 3-chloro-6-(2,2-difluoroethoxy)-4,5-dimethylpyridazine

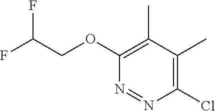

To a stirred solution of 3,6-dichloro-4,5-dimethylpyridazine (0.20 g, 1.1 mmol) in THF (10 mL) was added NaH (0.09 g, 2.2 mmol) at 0° C. The resulting reaction mixture was stirred for 10 min before 2,2-difluoroethan-1-ol (0.09 g, 1.1 mmol) was added. The reaction mixture was stirred at 90° C. for 16 h and the reaction was quenched by addition of ice water (10 mL) and extracted with EtOAc (3×20 mL), washed with water (20 mL). The combined organic layers were dried over anhydrous Na₂SO₄, filtered and concentrated in vacuo. The crude product was purified by column chromatography (SiO₂, 0-7% EtOAc/PE) to afford the title compound (0.19 g, 75%) as solid. LCMS m/z=223.1 [M+H]⁺.

### Synthesis of INT_034: 5-(difluoromethoxy)-2-iodo-3-methylpyridine

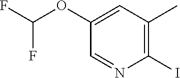

CuI (0.29 g, 1.5 mmol) and DMEDA (133 mg, 1.5 mmol) were added to a stirred solution of 2-bromo-5-(difluoromethoxy)-3-methylpyridine (INT 008, 0.36 g, 1.5 mmol, 1.0 equiv.) and NaI (1.13 g, 7.6 mmol, 5.0 equiv.) in 1,4-dioxane (12 mL) at room temperature under argon in a high pressure tube. The tube was sealed and the reaction mixture stirred at 120° C. for 18 h before being cooled to room temperature, filtered through a pad of celite and washed with 10% MeOH in EtOAc (2×25 mL).

The filtrate was washed with water (30 mL), dried over anhydrous Na₂SO₄, filtered and concentrated in vacuo. The crude product was purified by column chromatography (SiO₂, 0-30% EtOAc/PE) to afford the title compound (0.17 g, 41%) as a liquid. LCMS m/z=285.9 [M+H]⁺.

### Synthesis of Exemplary Compounds

### Synthesis of EX-I-01: cis-3-(5-cyclopropoxy-3-methylpyridin-2-yl)-8-(3,5-difluorophenyl)-8-(dimethylamino)-1,3-diazaspiro[4.5]decan-2-one

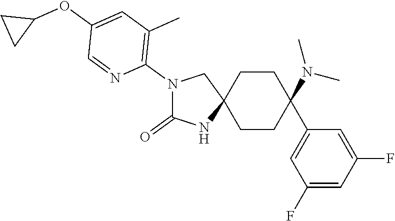 [SMILES: Cc1cc(OC2CC2)cnc1N1C[C@]2(CC[C@](c3cc(F)cc(F)c3)(N(C)C)CC2)NC1=O]

A mixture of 2-bromo-5-cyclopropoxy-3-methylpyridine (INT_005, 7.5 g, 32.9 mmol) and cis-8-(3,5-difluorophenyl)-8-(dimethylamino)-1,3-diazaspiro[4.5]decan-2-one (INT_004, 12.3 g, 29.9 mmol) and K₃PO₄ (19 g, 89.7 mmol) in dioxane (600 L) was stirred at 25° C. for 30 min. To this was added CuI (5.69 g, 29.9 mmol), DMEDA (5.27 g, 59.8 mmol) and the mixture stirred at 120° C. for 16 h. The solids were removed by filtration and the filter cake washed with MTBE (80 mL) and the filtrate extracted with EtOAc (300 mL×2) washed with H₂O (1000 mL). The combined organics were washed with brine (100 mL×2), dried (Na₂SO₄) and evaporated to dryness in vacuo. The residue was stirred with MTBE at 15° C. for 30 min and the solids collected by filtration and washed with MTBE (50 mL). The solids were dried under reduced pressure and further purified by trituration. The solid was stirred with MeCN (260 mL) at 90° C. for 30 min, cooled to 50° C., diluted with MeCN (130 mL) and stirred at 90° C. for 30 min. The mixture was filtered and the filtrate cooled to 50° C., stirred at 90° C. for 10 min and then cooled to 15° C. The solids were collected by filtration and dried under reduced pressure to afford the title compound as an oil (23 g, 83.4%). LCMS m/z=457 [M+H]⁺; ¹H NMR (400 MHz, DMSO-d₆) δ: 7.99 (d, 1H), 7.39 (d, 1H), 7.28 (br s, 1H), 7.13 (br t, 1H), 7.05 (br d, 2H), 3.91 (tt, 1H), 3.58 (d, 2H), 2.39-2.26 (m, 2H), 2.23 (s, 3H), 1.96 (s, 6H), 1.94-1.69 (m, 4H), 1.49 (br s, 2H), 0.83-0.64 (m, 4H).

### Synthesis of EX-I-02: cis-8-(3,5-difluorophenyl)-8-(dimethylamino)-3-(2-methoxy-4-methylpyrimidin-5-yl)-1,3-diazaspiro[4.5]decan-2-one

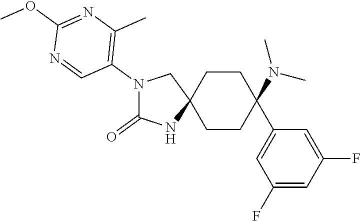 [SMILES: COc1ncc(N2C[C@]3(CC[C@](c4cc(F)cc(F)c4)(N(C)C)CC3)NC2=O)c(C)n1]

To a solution of cis-8-(3,5-difluorophenyl)-8-(dimethylamino)-1,3-diazaspiro[4.5]decan-2-one (INT_004, 5.4 g, 17.45 mmol) in dioxane (70 mL) in a sealed tube were added 5-bromo-2-methoxy-4-methylpyrimidine (INT_006, 3.54 g, 17.45 mmol) and K₂CO₃ (7.2 g, 52.35 mmol). The mixture was degassed for 15 min with argon and then DMEDA (0.52 mL, 3.49 mmol) and CuI (0.332 g, 1.745 mmol) were added and the mixture was stirred at 130° C. for 16 h. A second batch was carried out using above procedure with 7 g of INT_004. After the reactions were complete the mixtures were combined and filtered through a pad of celite. The filtrate was concentrated in vacuo, diluted with 10% MeOH-DCM (400 mL), washed with water (2×150 mL) and brine (150 mL). The combined organics were dried (Na₂SO₄) and evaporated to dryness in vacuo. The residue was purified by combi-flash chromatography (0-10% MeOH/DCM) to afford the title compound as a solid (9.2 g, 53%). LCMS m/z=432 [M+H]⁺; ¹H NMR (400 MHz, DMSO-d₆) δ: 8.38 (s, 1H), 7.34 (brs, 1H), 7.14-7.10 (m, 1H), 7.05 (d, 2H), 3.87 (s, 3H), 3.47 (s, 2H), 2.45-2.30 (m, 5H), 1.96 (s, 6H), 1.93-1.79 (m, 4H), 1.53-1.51 (m, 2H).

### Synthesis of EX-I-03: cis-8-(3,5-difluorophenyl)-8-(dimethylamino)-3-(5-(2-hydroxypropan-2-yl)-3-methylpyridin-2-yl)-1,3-diazaspiro[4.5]decan-2-one

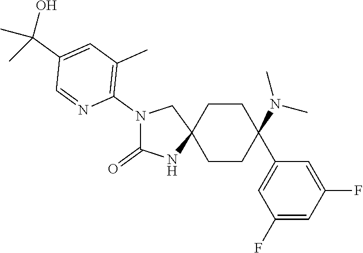 [SMILES: Cc1cc(C(C)(C)O)cnc1N1C[C@]2(CC[C@](c3cc(F)cc(F)c3)(N(C)C)CC2)NC1=O]

Pd₂(dba)₃ (11.85 g, 13 mmol) and Xantphos (15.0 g, 25.8 mmol) were added to a mixture of cis-8-(3,5-difluorophenyl)-8-(dimethylamino)-1,3-diazaspiro[4.5]decan-2-one (INT_004, 40 g, 129 mmol), 2-(6-chloro-5-methylpyridin-3-yl)propan-2-ol (INT_007, 29 g, 155 mmol) and Cs₂CO₃ (127 g, 388 mmol) in 1,4-dioxane (1 L) at room temperature under argon. The mixture was stirred at 120° C. for 16 h before it was allowed to cool to room temperature and filtered through a pad of celite. The cake was washed with EtOAc (2×500 mL) and the combined organic layers were concentrated under reduced pressure. The crude product was purified by combi-flash chromatography (5% MeOH/DCM) and the product was recrystallized from MeCN/H₂O 4:1 (840 mL, 80° C.→room temperature) to yield the title compound as a solid (25 g, 43%). LCMS m/z=459 [M+H]⁺; ¹H NMR (400 MHz, DMSO-d₆) δ: 8.27 (d, 1H), 7.66 (d, 1H), 7.31 (br s, 1H), 7.16-7.01 (m, 3H), 5.13 (s, 1H), 3.66-3.55 (m, 2H), 2.37-2.17 (m, 5H), 2.01-1.77 (m, 10H), 1.57-1.38 (m, 8H).

### Synthesis of EX-I-04: cis-3-(5-(difluoromethoxy)-3-methylpyridin-2-yl)-8-(3,5-difluorophenyl)-8-(dimethylamino)-1,3-diazaspiro[4.5]decan-2-one

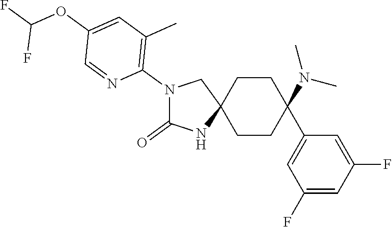 [SMILES: Cc1cc(OC(F)F)cnc1N1C[C@]2(CC[C@](c3cc(F)cc(F)c3)(N(C)C)CC2)NC1=O]

K₂CO₃ (38.2 g, 276 mmol), CuI (11.7 g, 61.4 mmol) and DMEDA (10.8 g, 123 mmol) were added to a mixture of cis-8-(3,5-difluorophenyl)-8-(dimethylamino)-1,3-diazaspiro[4.5]decan-2-one (INT_004, 19 g, 61.4 mmol), 2-bromo-5-(difluoromethoxy)-3-methylpyridine (INT_008, 20.4 g, 85.7 mmol) in 1,4-dioxane (200 mL) at 20° C. and the resulting mixture stirred at 120° C. for 16 h. The reaction mixture was filtered and washed with EtOAc (200 mL). The filtrate was diluted with water (50 mL) and extracted with EtOAc (3×100 mL). The combined organics were washed with brine (50 mL), and evaporated to dryness in vacuo. The residue was purified by re-crystallization from MeCN (286 mL) to give the title compound as a solid (15.9 g, 55.5%). LCMS m/z=467 [M+H]⁺; ¹H NMR (400 MHz, DMSO-d₆) δ: 8.12 (d, 1H), 7.57 (d, 1H), 7.47-7.40 (m, 1H), 7.26-7.01 (m, 4H), 3.65 (s, 2H), 2.34-2.25 (m, 5H), 1.98-1.83 (m, 10H), 1.52 (br d, 2H).

### Synthesis of EX-I-05: cis-8-(3,5-difluorophenyl)-8-(dimethylamino)-3-(4-methyl-1H-pyrrolo[2,3-c]pyridin-5-yl)-1,3-diazaspiro[4.5]decan-2-one

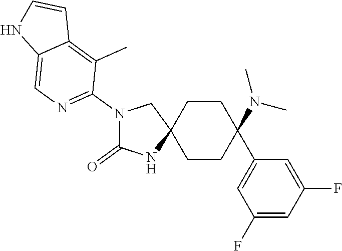 [SMILES: Cc1c(N2C[C@]3(CC[C@](c4cc(F)cc(F)c4)(N(C)C)CC3)NC2=O)ncc2[nH]ccc12]

To a stirred solution of cis-8-(3,5-difluorophenyl)-8-(dimethylamino)-3-(4-methyl-1-{[2-(trimethylsilyl)ethoxy]methyl}-1H-pyrrolo[2,3-c]pyridin-5-yl)-1,3-diazaspiro[4.5]decan-2-one (INT_015, 0.2 g, 0.35 mmol) in DCM (5 ml) was added TFA (3 ml) at 0° C. and the reaction mixture was stirred at room temperature for 1 h. The reaction mixture was concentrated under reduced pressure and MeOH (5 ml) was added followed by aqueous NH₃ solution (25%, 4 ml) and stirred for another 1 h. The mixture was concentrated and the residue was purified by preparative HPLC to afford the title compound as a solid (0.09 g, 58%). LCMS m/z=440 [M+H]⁺; H NMR (400 MHz, DMSO-d₆, 100° C.) 6: 11.19 (bs, 1H), 8.41 (s, 1H), 7.50 (d, 1H), 6.97-7.02 (m, 3H), 6.69 (s, 1H), 6.50 (d, 1H), 3.64 (s, 2H), 2.30-2.73 (m, 5H), 2.06 (s, 6H), 1.85-2.01 (m, 4H), 1.55-1.60 (m, 2H).

### Synthesis of EX-I-06: cis-8-(3,5-difluorophenyl)-3-(5-(2-hydroxypropan-2-yl)-3-methylpyridin-2-yl)-8-(methylamino)-1,3-diazaspiro[4.5]decan-2-one

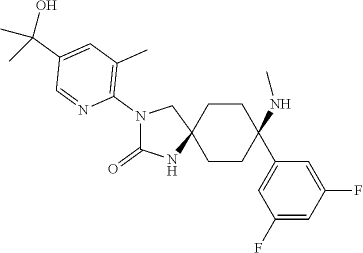 [SMILES: CN[C@]1(c2cc(F)cc(F)c2)CC[C@]2(CC1)CN(c1ncc(C(C)(C)O)cc1C)C(=O)N2]

To a solution of 2-(6-chloro-5-methylpyridin-3-yl)propan-2-ol (INT_007, 1.84 g, 9.8 mmol) in 1,4-dioxane (30 mL) were added cis-8-(3,5-difluorophenyl)-8-(methylamino)-1,3-diazaspiro[4.5]decan-2-one (INT_018, 2.4 g, 8.2 mmol), Xantphos (0.71 g, 1.2 mmol), Cs₂CO₃ (7.9 g, 2.5 mmol) and Pd₂(dba)₃ (1.1 g, 1.2 mmol) at room temperature under argon atmosphere. The resulting reaction mixture was stirred at 120° C. for 18 h. The reaction mixture was cooled to room temperature, filtered through pad of Celite and concentrated in vacuo. The residue was purified by preparative HPLC to yield the title compound as a colorless solid (2.0 g, 58%). That solid was dissolved in MeCN (45 mL) at room temperature and stirred at 90° C. for 30 min and subsequently at 60° C. for 30 min. The solution was allowed to cool to room temperature an left undisturbed for 18 h to afford the title compound as a solid (1.90 g, 95%) after collecting the material by filtration. LCMS m/z=445.4 [M+H]⁺; ¹H NMR (400 MHz, DMSO-d₆, 25° C.) δ: 8.29 (d, 1H), 7.67 (d, 1H), 7.18-7.15 (m, 2H), 7.04-6.99 (m, 2H), 5.14 (s, 1H), 3.76 (s, 2H), 2.29-2.27 (m, 1H), 2.23 (s, 3H), 2.06-2.00 (m, 2H), 1.89 (d, 3H), 1.82-1.70 (m, 4H), 1.60-1.57 (m, 2H), 1.43 (s, 6H).

### Synthesis of EX-I-07: cis-8-(3,5-difluorophenyl)-8-(dimethylamino)-3-(3-methyl-5-(trifluoromethoxy)pyridin-2-yl)-1,3-diazaspiro[4.5]decan-2-one

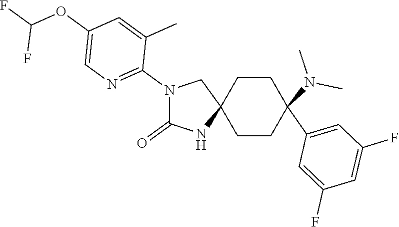 [SMILES: Cc1cc(OC(F)F)cnc1N1C[C@]2(CC[C@](c3cc(F)cc(F)c3)(N(C)C)CC2)NC1=O]

To a solution of cis-8-(3,5-difluorophenyl)-8-(dimethylamino)-1,3-diazaspiro[4.5]decan-2-one (INT_004, 1.2 g, 3.9 mmol) in 1,4-Dioxane (20 mL) were added 2-bromo-3-methyl-5-(trifluoromethoxy)pyridine (1.19 g, 4.66 mmol) and K₂CO₃ (1.6 g, 11.65 mmol) in a sealed tube under argon. Trans-N,N′-dimethylcyclohexane-1,2-diamine (0.12 mL, 0.78 mmol) and CuI (0.074 g, 0.388 mmol) were added and the resulting mixture was stirred at 130° C. for 16 h. The reaction mixtures was cooled to room temperature and filtered through a pad of celite. The filtrate was concentrated under reduced pressure, the residue was diluted with 10% MeOH—CH₂Cl₂ (150 mL) and washed with water (2×50 mL) and sat. NaCl solution (50 mL). The combined organic layers were dried over anhydrous Na₂SO₄ and concentrated under reduced pressure. The crude product which was purified by combi-flash chromatography (2-4% MeOH in DCM as eluent) followed by preparative HPLC purification to afford the title compound which was crystallized from MeCN as described for EX-I-04 to afford the title compound as a solid (1.15 g, 61%). LCMS m/z=485.3 [M+H]⁺; ¹H NMR (400 MHz, DMSO-d₆, 100° C.) 6: 8.24 (s, 1H), 7.69 (s, 1H), 7.20 (s, 1H), 6.98-7.01 (m, 3H), 3.69 (s, 2H), 2.29-2.34 (m, 5H), 2.05 (s, 6H), 1.83-1.99 (m, 4H), 1.53-1.58 (m, 2H).

### Synthesis of EX-I-08: cis-8-(3,5-difluorophenyl)-8-(dimethylamino)-3-(4-methyl-6-(trifluoromethoxy)pyridazin-3-yl)-1,3-diazaspiro[4.5]decan-2-one hydrochloride

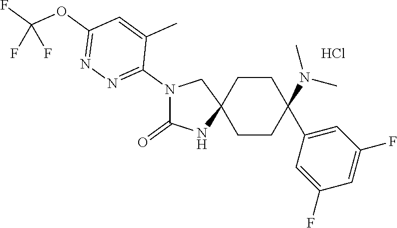 [SMILES: Cc1cc(OC(F)(F)F)nnc1N1C[C@]2(CC[C@](c3cc(F)cc(F)c3)(N(C)C)CC2)NC1=O ; Cl]

The free base of the title compound (0.17 g, 30%) was prepared in analogy to EX-I-06 from INT_004 (0.36 g, 1.17 mmol) and 3-bromo-4-methyl-6-(trifluoromethoxy)pyridazine (0.3 g, 1.40 mmol). A suspension of the product (0.1 g, 0.21 mmol) in Et₂O (5 mL) was treated with HCl (2 M in Et₂O, 0.51 mL, 1.03 mmol) and gently warmed to obtain a solution. The solvent was removed under reduced pressure to afford the title compound as a solid (0.07 g, 65%). LCMS m/z=486.5 [M+H]⁺; ¹H NMR (400 MHz, DMSO-d₆, 25° C.) 6: 10.1 (s, 1H), 8.21 (s, 1H), 7.68 (s, 1H), 7.46-7.52 (m, 3H), 3.71 (s, 2H), 2.69 (d, 2H), 2.59-2.60 (m, 5H), 2.35 (s, 3H), 2.23-2.30 (m, 2H), 1.93 (d, 2H), 1.39 (t, 2H).

### Synthesis of EX-I-09: cis-8-(3,5-difluorophenyl)-8-(dimethylamino)-3-(5-(3-hydroxy-3-methylcyclobutoxy)-3-methylpyridin-2-yl)-1,3-diazaspiro[4.5]decan-2-one

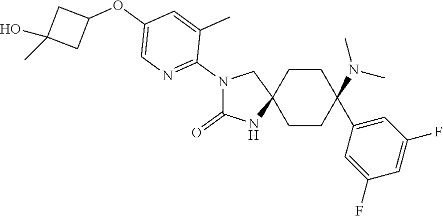 [SMILES: Cc1cc(OC2CC(C)(O)C2)cnc1N1C[C@]2(CC[C@](c3cc(F)cc(F)c3)(N(C)C)CC2)NC1=O]

The title compound (33 mg, 10%) was prepared in analogy to EX-I-01 from INT_004 (0.21 g, 0.78 mmol) and 3-((6-bromo-5-methylpyridin-3-yl)oxy)-1-methylcyclobutan-1-ol (0.2 g, 0.65 mmol). LCMS m/z=501.2 [M+H]⁺; ¹H NMR (400 MHz, DMSO-d₆, 25° C.) 6: 7.81 (d, 1H), 7.24 (br s, 1H), 7.17 (d, 1H), 7.14-7.10 (m, 1H), 7.04 (s, 1H), 7.02 (s, 1H), 5.14 (s, 1H), 4.44-4.37 (m, 1H), 3.57 (s, 2H), 2.55-2.52 (m, 2H), 2.32-2.29 (m, 2H), 2.19 (s, 3H), 2.08-2.03 (m, 2H), 1.96 (s, 6H), 1.88-1.86 (m, 4H), 1.48-1.46 (m, 2H), 1.25 (s, 3H).

### Synthesis of EX-I-10: cis-8-(3,5-difluorophenyl)-8-(dimethylamino)-3-(3-methyl-5-(methylsulfonyl)pyridin-2-yl)-1,3-diazaspiro[4.5]decan-2-one

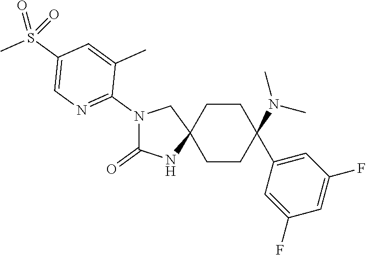 [SMILES: Cc1cc(S(C)(=O)=O)cnc1N1C[C@]2(CC[C@](c3cc(F)cc(F)c3)(N(C)C)CC2)NC1=O]

The title compound (0.33 g, 21%) was prepared in analogy to EX-I-06 from INT_004 (1.0 g, 3.23 mmol) and 2-chloro-3-methyl-5-(methylsulfonyl)pyridine (1.0 g, 4.85 mmol). LCMS m/z=479.2 [M+H]⁺; ¹H NMR (400 MHz, DMSO-d₆, 25° C.) 6: 8.65 (d, 1H), 8.14 (d, 1H), 7.72 (br s, 1H), 7.13 (br t, 1H), 7.04 (br d, 2H), 3.75 (s, 2H), 3.27 (s, 3H), 2.34 (s, 5H), 1.96 (s, 6H), 1.94-1.79 (m, 4H), 1.52 (br s, 2H).

### Synthesis of EX-I-11: cis-8-(3,5-difluorophenyl)-8-(dimethylamino)-3-(5-(ethylsulfonyl)-3-methylpyridin-2-yl)-1,3-diazaspiro[4.5]decan-2-one

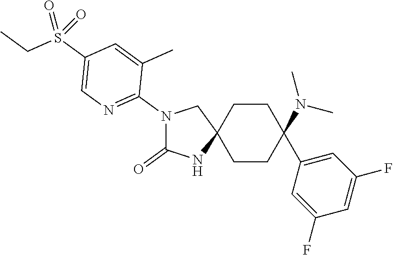 [SMILES: CCS(=O)(=O)c1cnc(N2C[C@]3(CC[C@](c4cc(F)cc(F)c4)(N(C)C)CC3)NC2=O)c(C)c1]

The title compound (0.15 g, 46%) was prepared in analogy to EX-I-07 from INT_004 (0.2 g, 0.65 mmol) and 2-bromo-5-(ethylsulfonyl)-3-methylpyridine (0.17 g, 0.65 mmol). LCMS m/z=493.3 [M+H]⁺; ¹H NMR (400 MHz, DMSO-d₆, 25° C.) 6: 8.60 (d, 1H), 8.09 (d, 1H), 7.72 (s, 1H), 7.12 (t, 1H), 7.04 (d, 2H), 3.76 (s, 2H), 3.35-3.37 (m, 2H), 2.30-2.40 (m, 5H), 1.79-1.96 (m, 11H), 1.52-1.54 (m, 2H), 1.11 (t, 3H).

### Synthesis of EX-I-12: cis-8-(3,5-difluorophenyl)-8-(dimethylamino)-3-(2-methyl-6-(trifluoromethyl)pyridin-3-yl)-1,3-diazaspiro[4.5]decan-2-one

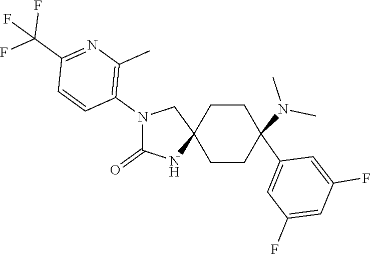 [SMILES: Cc1nc(C(F)(F)F)ccc1N1C[C@]2(CC[C@](c3cc(F)cc(F)c3)(N(C)C)CC2)NC1=O]

The title compound (95 mg, 44%) was prepared in analogy to EX-I-01 from INT_004 (0.14 g, 0.45 mmol) and 3-bromo-2-methyl-6-(trifluoromethyl)pyridine (119 mg, 0.50 mmol). LCMS m/z=469.4 [M+H]⁺; ¹H NMR (400 MHz, DMSO-d₆, 25° C.) 6: 7.88 (d, 1H), 7.73 (d, 1H), 7.52 (brs, 1H), 7.15-7.10 (s, 1H), 7.06-7.04 (m, 2H), 3.60 (s, 2H), 2.46 (s, 3H), 2.40-2.30 (m, 2H), 1.97-1.80 (m, 10H), 1.53-1.49 (m, 2H).

### Synthesis of EX-I-13: cis-8-(3,5-difluorophenyl)-8-(dimethylamino)-3-(2,6-dimethylpyridin-3-yl)-1,3-diazaspiro[4.5]decan-2-one

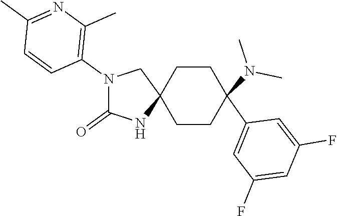 [SMILES: Cc1ccc(N2C[C@]3(CC[C@](c4cc(F)cc(F)c4)(N(C)C)CC3)NC2=O)c(C)n1]

The title compound (40 mg, 15%) was prepared in analogy to EX-I-07 from INT_004 (0.20 g, 0.65 mmol) and 3-bromo-2,6-dimethylpyridine (144 mg, 0.78 mmol). LCMS m/z=415.4 [M+H]⁺; 1H NMR (400 MHz, DMSO-d₆, 100° C.) 6: 7.43 (d, 1H), 7.0-7.04 (m, 4H), 6.89 (s, 1H), 3.45 (s, 2H), 2.41 (s, 3H), 2.18-2.34 (m, 5H), 2.06 (s, 6H), 1.85-1.98 (m, 4H), 1.52-1.56 (m, 2H).

### Synthesis of EX-I-14: cis-8-(3,5-difluorophenyl)-8-(dimethylamino)-3-(2-methyl-6-(trifluoromethoxy)pyridin-3-yl)-1,3-diazaspiro[4.5]decan-2-one hydrochloride

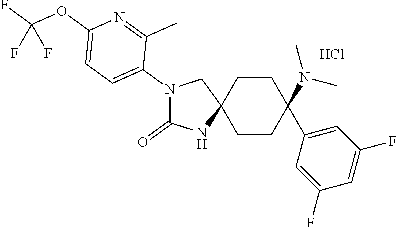 [SMILES: Cc1nc(OC(F)(F)F)ccc1N1C[C@]2(CC[C@](c3cc(F)cc(F)c3)(N(C)C)CC2)NC1=O ; Cl]

The free base of the title compound (0.11 g, 17%) was prepared in analogy to EX-I-06 from INT_004 (0.40 g, 1.29 mmol) and 3-bromo-2-methyl-6-(trifluoromethoxy)pyridine (0.40 g, 1.55 mmol). A suspension of the product (0.11 g, 0.22 mmol) in H₂O (5 mL) was treated with HCl (1 M in H₂O, 0.32 mL, 0.22 mmol) and gently warmed to obtain a solution. The solvent was removed under reduced pressure to afford the title compound as a solid (0.07 g, 65%). LCMS m/z=10.49 (s, 1H), 7.84 (d, 1H), 7.73 (s, 1H), 7.54 (d, 2H), 7.44 (t, 1H), 7.14 (d, 1H), 3.39 (s, 2H), 2.70 (d, 2H), 2.58 (d, 6H), 2.28-2.33 (m, 5H), 1.91 (d, 2H), 1.33 (t, 2H).

### Synthesis of EX-I-15: cis-8-(3,5-difluorophenyl)-8-(dimethylamino)-3-(4-methyl-2-(oxetan-3-yloxy)pyrimidin-5-yl)-1,3-diazaspiro[4.5]decan-2-one

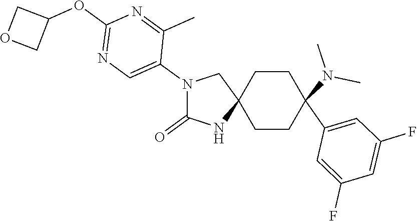 [SMILES: Cc1nc(OC2COC2)ncc1N1C[C@]2(CC[C@](c3cc(F)cc(F)c3)(N(C)C)CC2)NC1=O]

The title compound (88 mg, 29%) was prepared in analogy to EX-I-07 from INT_004 (0.20 g, 0.65 mmol) and 5-bromo-4-methyl-2-(oxetan-3-yloxy)pyrimidine (191 mg, 0.78 mmol). LCMS m/z=474.2 [M+H]⁺; ¹H NMR (400 MHz, DMSO-d₆, 25° C.) 6: 8.38 (s, 1H), 7.36-7.43 (m, 1H), 7.03-7.12 (m, 3H), 5.49-5.52 (m, 1H), 4.84-4.88 (m, 2H), 4.53-4.56 (m, 2H), 3.47 (s, 2H), 2.29-2.37 (m, 5H), 1.80-1.96 (m, 10H), 1.36-1.48 (m, 2H).

### Synthesis of EX-I-16: cis-8-(3,5-difluorophenyl)-8-(dimethylamino)-3-(2-(2-methoxyethoxy)-4-methylpyrimidin-5-yl)-1,3-diazaspiro[4.5]decan-2-one

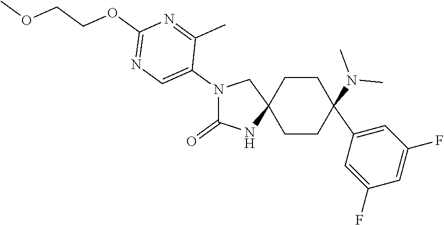 [SMILES: COCCOc1ncc(N2C[C@]3(CC[C@](c4cc(F)cc(F)c4)(N(C)C)CC3)NC2=O)c(C)n1]

The title compound (65 mg, 27%) was prepared in analogy to EX-I-01 from INT_004 (0.20 g, 0.64 mmol) and 5-bromo-2-(2-methoxyethoxy)-4-methylpyrimidine (322 mg, 1.29 mmol). LCMS m/z=476.2 [M+H]⁺; ¹H NMR (400 MHz, DMSO-d₆, 25° C.) 6: 8.37 (s, 1H), 7.34 (br s, 1H), 7.15-7.09 (m, 2H), 7.06-7.03 (m, 2H), 4.39-4.36 (m, 2H), 3.65-3.62 (m, 2H), 3.47-3.45 (br s, 2H), 3.28 (s, 3H), 2.33-2.29 (m, 5H), 1.96-1.80 (m, 9H), 1.50 (br s, 2H).

### Synthesis of EX-I-17: cis-8-(3,5-difluorophenyl)-8-(dimethylamino)-3-(2-methyl-6-(methylsulfonyl)pyridin-3-yl)-1,3-diazaspiro[4.5]decan-2-one

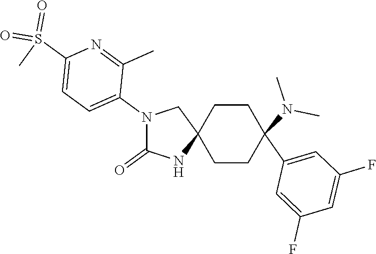 [SMILES: Cc1nc(S(C)(=O)=O)ccc1N1C[C@]2(CC[C@](c3cc(F)cc(F)c3)(N(C)C)CC2)NC1=O]

The title compound (0.13 g, 56%) was prepared in analogy to EX-I-06 from INT_004 (0.15 g, 0.49 mmol) and 3-bromo-2-methyl-6-(methylsulfonyl)pyridine (0.18 g, 0.73 mmol). LCMS m/z=479.2 [M+H]⁺; ¹H NMR (400 MHz, DMSO-d₆, 25° C.) δ: 7.93 (d, 1H), 7.87 (d, 1H), 7.57 (br s, 1H), 7.16-7.08 (m, 1H), 7.07-7.02 (m, 2H), 3.61 (s, 2H), 3.24 (s, 3H), 2.49 (s, 3H), 2.46-2.28 (m, 2H), 1.96-1.70 (m, 10H), 1.60-1.48 (m, 2H).

### Synthesis of EX-I-18: cis-3-(2-cyclopropoxy-4-methylpyrimidin-5-yl)-8-(3,5-difluorophenyl)-8-(dimethylamino)-1,3-diazaspiro[4.5]decan-2-one

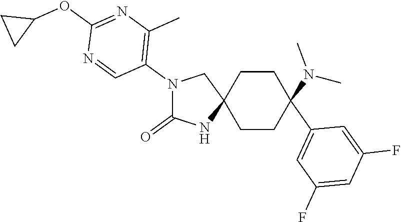 [SMILES: Cc1nc(OC2CC2)ncc1N1C[C@]2(CC[C@](c3cc(F)cc(F)c3)(N(C)C)CC2)NC1=O]

A mixture of 5-bromo-2-cyclopropoxy-4-methylpyrimidine (INT_019, 19 g, 82.9 mmol) and cis-8-(3,5-difluorophenyl)-8-(dimethylamino)-1,3-diazaspiro[4.5]decan-2-one (INT_004, 23.3 g, 75.4 mmol) and K₃PO₄ (48 g, 226 mmol) in 1,4-dioxane (1 L) was stirred at 25° C. for 30 min. To this was added CuI (14.4 g, 75.4 mmol), DMEDA (13.3 g, 151 mmol) and the mixture stirred at 120° C. for 16 h. The solids were removed by filtration and washed with EtOAc (100 mL). The filtered was extracted with EtOAc (500 mL×3) and the combined organics evaporated to dryness under reduced pressure and the residue purified by column chromatography (SiO₂, 9-100% MeOH/DCM). The residue was further purified by trituration with MeCN (260 ml) at 90° C. for 30 min, cooled to 50° C. and further MeCN (13 ml) added at 50° C. The mixture was stirred at 90° C. for 30 mins, filtered, cooled to 50° C., stirred at 90° C. for 10 min and then stirred at 25° C. for 16 h. The solids were collected and dried under reduced pressure. The resulting solid was triturated with MTBE at 25° C. for 2 h and the solids collected by filtration and the solids dried under reduced pressure. The solids were stirred with MeCN (260 ml) at 90° C. for 30 min, cooled to 50° C., diluted with MeCN (13.0 mL) at 50° C. and stirred at 90° C. for 30 min. The hot solution was filtered, cooled to 50° C., warmed to 90° C. for 10 min and then stirred at 25° C. for 16 h. The solids were collected by filtration and dried under reduced pressure to afford the title compound as a solid (18.1 g, 52%). LCMS m/z=458 [M+H]⁺; ¹H NMR (400 MHz, DMSO-d₆) δ: 8.39 (s, 1H), 7.36 (br s, 1H), 7.12 (br t, 1H), 7.05 (br d, 2H), 4.25 (tt, 1H), 3.48 (s, 2H), 2.42-2.22 (m, 5H), 2.03-1.69 (m, 10H), 1.57-1.42 (m, 2H), 0.78-0.72 (m, 2H), 0.71-0.65 (m, 2H).

### Synthesis of EX-I-19: cis-8-(3,5-difluorophenyl)-3-(1,3-dimethyl-2-oxo-1,2-dihydropyridin-4-yl)-8-(dimethylamino)-1,3-diazaspiro[4.5]decan-2-one

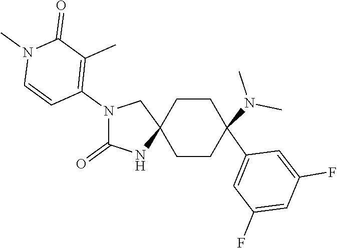 [SMILES: Cc1c(N2C[C@]3(CC[C@](c4cc(F)cc(F)c4)(N(C)C)CC3)NC2=O)ccn(C)c1=O]

K₃PO₄ (13.4 g, 63.1 mmol) was added to an argon degassed solution of cis-8-(3,5-difluorophenyl)-8-(dimethylamino)-1,3-diazaspiro[4.5]decan-2-one (INT_004, 6.5 g, 21.0 mmol) and 4-bromo-1,3-dimethylpyridin-2(1H)-one (INT_020, 5.1 g, 25.2 mmol) in dioxane (200 mL) at rt. To the resulting suspension was added sequentially CuI (4.01 g, 21 mmol) and DMEDA (2.26 mL, 21.0 mmol) and the reaction mixture degassed for 10 min and then heated at 120° C. for 18 h. The reaction mixture was cooled to rt, filtered through celite pad and washed with EtOAc (300 mL). The filtrate was diluted with water (800 mL) and the organic layer was separated. The combined organics were dried (Na₂SO₄) and evaporated to dryness in vacuo to afford the title compound as a white solid (9.8 g, crude). The solid in acetonitrile (350 mL) was heated to 90° C. for 2.5 h. The resulting clear solution was then stirred at 60° C. for 30 min and then allowed to slowly cool to rt and stirred for 18 h. The solid was collected by filtration and dried under reduced pressure to afford the title compound as a solid (5.5 g). The filtrate evaporated under reduced pressure to afford an additional 1.7 g of the title compound as a solid. Total yield: 7.2 g, 80%). LCMS m/z=431 [M+H]⁺; ¹H NMR (500 MHz, DMSO-d₆) δ: 7.48 (d, 1H), 7.37 (br s, 1H), 7.14-7.10 (m, 1H), 7.05-7.03 (m, 2H), 6.18 (d, 1H), 3.46 (s, 2H), 3.38 (s, 3H), 2.40-2.22 (m, 2H), 1.95-1.70 (m, 13H), 1.57-1.46 (m, 2H).

### Synthesis of EX-I-20: cis-8-(3,5-difluorophenyl)-8-(dimethylamino)-3-(5-(2-hydroxypropan-2-yl)-3-methylpyrazin-2-yl)-1,3-diazaspiro[4.5]decan-2-one

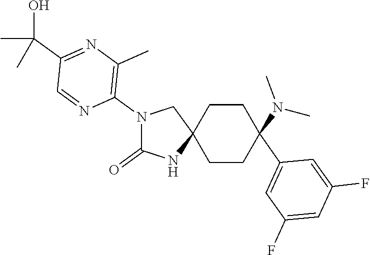 [SMILES: Cc1nc(C(C)(C)O)cnc1N1C[C@]2(CC[C@](c3cc(F)cc(F)c3)(N(C)C)CC2)NC1=O]

The title compound (1.4 g, 78%) was prepared in analogy to EX-I-06 from INT_004 (1.2 g, 3.9 mmol) and 2-(5-chloro-6-methylpyrazin-2-yl)propan-2-ol (INT_021, 0.87 g, 4.7 mmol). LCMS m/z=460 [M+H]⁺; ¹H NMR (400 MHz, DMSO-d₆) δ: 8.45 (s, 1H), 7.54 (brs, 1H), 7.12 (t, 1H), 7.04 (d, 2H), 5.32 (s, 1H), 3.68 (s, 2H), 2.43 (s, 3H), 2.32 (m, 2H), 1.96 (s, 6H), 1.88-1.93 (m. 4H), 1.51 (m, 2H), 1.43 (s, 6H).

### Synthesis of EX-I-21: cis-8-(3,5-difluorophenyl)-8-(dimethylamino)-3-(2-(3-fluorooxetan-3-yl)-4-methylpyrimidin-5-yl)-1,3-diazaspiro[4.5]decan-2-one

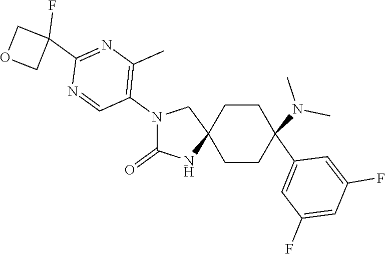 [SMILES: Cc1nc(C2(F)COC2)ncc1N1C[C@]2(CC[C@](c3cc(F)cc(F)c3)(N(C)C)CC2)NC1=O]

The title compound (130 mg, 45%) was prepared in analogy to EX-I-01 from INT_004 (0.21 g, 0.67 mmol) and 5-bromo-2-(3-fluorooxetan-3-yl)-4-methylpyrimidine (INT_024, 150 mg, 0.61 mmol). LCMS m/z=475 [M+H]⁺; H NMR (500 MHz, DMSO-d₆) δ: 8.73 (s, 1H), 7.56 (brs, 1H), 7.15-7.11 (m, 1H), 7.07-7.05 (m, 2H), 5.09-5.04 (m, 2H), 4.95-4.89 (m, 2H), 3.62 (s, 2H), 2.45 (s, 3H), 2.37-2.32 (m, 2H), 1.97-1.84 (m, 10H), 1.54-1.48 (m, 2H).

### Synthesis of EX-I-22: cis-8-(3,5-difluorophenyl)-8-(dimethylamino)-3-(2-(2-hydroxypropan-2-yl)-4-methylpyrimidin-5-yl)-1,3-diazaspiro[4.5]decan-2-one

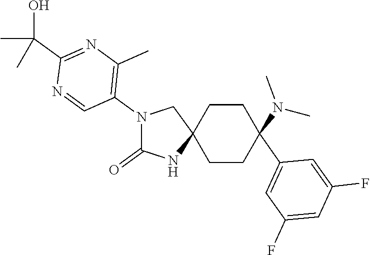 [SMILES: Cc1nc(C(C)(C)O)ncc1N1C[C@]2(CC[C@](c3cc(F)cc(F)c3)(N(C)C)CC2)NC1=O]

The title compound (130 mg, 45%) was prepared in analogy to EX-I-01 from INT_004 (0.26 g, 0.85 mmol) and 2-(5-bromo-4-methylpyrimidin-2-yl)propan-2-ol (INT_027, 236 mg, 1.02 mmol). LCMS m/z=460 [M+H]⁺; H NMR (400 MHz, DMSO-d₆) δ: 8.55 (s, 1H), 7.15 (s, 1H), 7.02-7.00 (m, 3H), 4.63 (s, 1H), 3.57 (s, 2H), 2.43 (s, 3H), 2.36-2.31 (m, 2H), 2.07 (s, 6H), 2.01-1.95 (m, 2H), 1.88-1.85 (m, 2H), 1.60-1.55 (m, 2H), 1.50 (s, 6H).

### Synthesis of EX-I-23: cis-3-(5-(1,1-difluoroethoxy)-1-methyl-6-oxo-1,6-dihydropyridin-2-yl)-8-(3,5-difluorophenyl)-8-(dimethylamino)-1,3-diazaspiro[4.5]decan-2-one

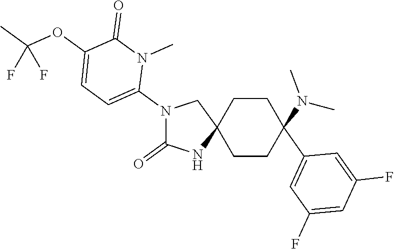 [SMILES: Cn1c(N2C[C@]3(CC[C@](c4cc(F)cc(F)c4)(N(C)C)CC3)NC2=O)ccc(OC(C)(F)F)c1=O]

6-chloro-3-(1,1-difluoroethoxy)-1-methylpyridin-2(1H)-one (INT_029, 1.62 g, 6.5 mmol) and Cs₂CO₃ (5.7 g, 17.6 mmol) were added to a solution of cis-8-(3,5-difluorophenyl)-8-(dimethylamino)-1,3-diazaspiro[4.5]decan-2-one (INT_004, 1.8 g, 5.9 mmol) in dioxane (100 mL). To this was added Xanthphos (509 g, 0.9 mmol) and Pd₂(dba)₃ (805 mg, 0.9 mmol) and the solution was stirred at 120° C. for 16 h under nitrogen atmosphere. The reaction was quenched by addition of H₂O (60 mL) and the mixture was extracted with EtOAc (3×50 mL). The combined organic layers were dried over anhydrous Na₂SO₄ and concentrated in vacuo. The residue was purified by column chromatography (SiO₂, 1-100% EtOAc/PE) to yield the title compound as a solid (1.8 g, 62%). LCMS m/z=497 [M+H]⁺; ¹H NMR (400 MHz, DMSO-d₆) δ: 1.52 (br d, 2H) 1.71-2.02 (m, 13H) 2.21-2.39 (m, 2H) 3.38 (s, 3H) 3.55 (s, 2H) 6.20 (d, 1H) 7.01-7.08 (m, 2H) 7.13 (brt, 1H) 7.36 (d, 1H) 7.73 (br s, 1H).

### Synthesis of EX-I-24: 8-(3,5-difluorophenyl)-8-(dimethylamino)-3-(3-methyl-5-((S)-1,1,1-trifluoro-2-hydroxypropan-2-yl)pyridin-2-yl)-1,3-diazaspiro[4.5]decan-2-one (enantiomer—2)

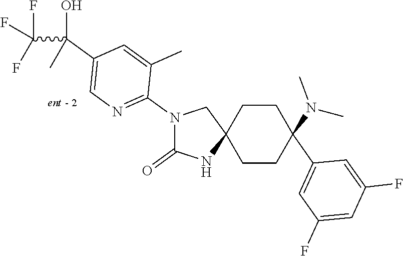 [SMILES: Cc1cc(C(C)(O)C(F)(F)F)cnc1N1C[C@]2(CC[C@](c3cc(F)cc(F)c3)(N(C)C)CC2)NC1=O]

In a sealed tube under argon atmosphere, Xantphos (70 mg, 0.12 mmol) and Pd₂(dba)₃ (110 mg, 0.12 mmol) were added to a stirred solution of cis-8-(3,5-difluorophenyl)-8-(dimethyl amino)-1,3-diazaspiro decan-2-one (INT_004, 0.25 g, 0.81 mmol), 2-(6-chloro-5-methylpyridin-3-yl)-1,1,1-trifluoropropan-2-ol (INT_031, 0.232 g, 0.970 mmol), and Cs₂CO₃ (0.788 g, 2.427 mmol) in 1,4-dioxane (20 mL) at room temperature. The reaction mixture was further degassed for 10 min by purging with argon. The resulting reaction mixture was heated to 120° C. and stirred for 16 h at that temperature. The reaction mixture was cooled to room temperature, filtered through a pad of celite which was washed with EtOAc (3×100 mL). The filtrate was concentrated in vacuo and the residue was purified by reverse phase column chromatography to yield the racemic compound as a solid (400 mg, 96%). The racemic material was separated by chiral SFC (Chiralpak IC 5 μM, 80% CO₂ 20% 0.5%-Et₂NH/MeOH 3 mL/min) to afford the single, secondly eluting enantiomer as a solid (90 mg). LCMS m/z=413 [M+H]⁺: ¹H NMR (400 MHz, DMSO-d₆) δ: 8.36 (d, 1H), 7.79 (d, 1H), 7.43 (brs, 1H), 7.15-7.09 (m, 1H), 7.05-7.03 (m, 2H), 6.74 (s, 1H), 3.67 (s, 2H), 2.33-2.26 (m, 5H), 1.96-1.87 (m, 10H), 1.69 (s, 3H), 1.52-1.48 (m, 2H).

### Synthesis of EX-I-25: cis-3-(5-cyclopropoxy-3-methylpyridin-2-yl)-8-(3,5-difluorophenyl)-8-(methylamino)-1,3-diazaspiro[4.5]decan-2-one

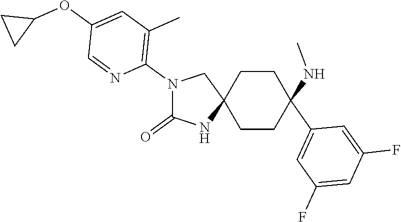 [SMILES: CN[C@]1(c2cc(F)cc(F)c2)CC[C@]2(CC1)CN(c1ncc(OC3CC3)cc1C)C(=O)N2]

The title compound was prepared from cis-8-(3,5-difluorophenyl)-8-(methylamino)-1,3-diazaspiro[4.5]decan-2-one (INT_018) and 2-bromo-5-cyclopropoxy-3-methylpyridine (INT_005) using an analogous method to that described for EX-I-01. The solid was crystallized from MeCN as described for EX-I-06 to afford the title compound as a solid (2.39 g, 64%). LCMS m/z=443.2 [M+H]⁺; H NMR (400 MHz, DMSO-d₆, 25° C.) δ: 7.99 (d, 1H), 7.38 (d, 1H), 7.17-7.14 (m, 2H), 7.04-6.98 (m, 2H), 3.94-3.90 (m, 1H), 3.72 (s, 2H), 2.32-2.23 (m, 4H), 2.06-2.00 (m, 2H), 1.90 (d, 3H), 1.82-1.57 (m, 6H), 0.81-0.77 (m, 2H), 0.68-0.64 (m, 2H).

### Synthesis of EX-I-26: cis-3-(5-(2,2-difluoroethoxy)-3-methylpyridin-2-yl)-8-(3,5-difluorophenyl)-8-(dimethylamino)-1,3-diazaspiro[4.5]decan-2-one

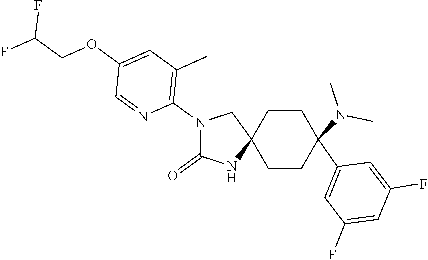 [SMILES: Cc1cc(OCC(F)F)cnc1N1C[C@]2(CC[C@](c3cc(F)cc(F)c3)(N(C)C)CC2)NC1=O]

The title compound (54 mg, 26%) was prepared in analogy to EX-I-06 from INT_004 (0.16 g, 0.52 mmol) and 2-chloro-5-(2,2-difluoroethoxy)-3-methylpyridine (INT_032, 0.12 g, 0.58 mmol). LCMS m/z=479.3 [M−H]⁻, H NMR (400 MHz, DMSO-d₆) δ: 7.98 (d, 1H), 7.39 (d, 1H), 7.28 (bs, 1H), 7.12 (t, 1H), 7.04-7.02 (m, 2H), 6.38 (tt, 1H), 4.37 (td, 2H), 3.59 (s, 2H), 2.32-2.22 (m, 5H), 1.96-1.86 (m, 10H), 1.51-1.49 (m, 2H).

### Synthesis of SC_027: cis-3-(6-(2,2-difluoroethoxy)-4,5-dimethylpyridazin-3-yl)-8-(3,5-difluorophenyl)-8-(dimethylamino)-1,3-diazaspiro[4.5]decan-2-one

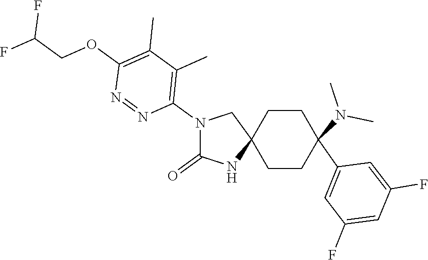 [SMILES: Cc1c(OCC(F)F)nnc(N2C[C@]3(CC[C@](c4cc(F)cc(F)c4)(N(C)C)CC3)NC2=O)c1C]

The title compound was prepared as a solid (135 mg, 40%) from cis-8-(3,5-difluorophenyl)-8-(dimethylamino)-1,3-diazaspiro[4.5]decan-2-one (INT_004, 0.20 g, 0.65 mmol) and 3-chloro-6-(2,2-difluoroethoxy)-4,5-dimethylpyridazine (INT_033, 0.17 g, 0.78 mmol)) using an analogous method to that described for EX-I-06. LCMS m/z=494.4 [M−H]⁻, ¹H NMR (400 MHz, DMSO-d₆) δ: 7.56 (br, 1H), 7.15-7.04 (m, 3H), 6.44 (tt, 1H), 4.69 (td, 2H), 3.71 (s, 2H), 2.32 (m, 2H), 2.16 (s, 6H), 1.96-1.89 (m, 10H), 1.52 (m, 2H).

### Synthesis of EX-I-28: cis-3-(5-(difluoromethoxy)-3-methylpyridin-2-yl)-8-(3,5-difluorophenyl)-8-(dimethylamino)-1,3-diazaspiro[4.5]decane-2,4-dione

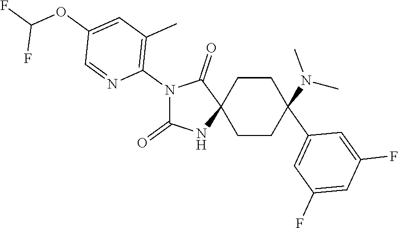 [SMILES: Cc1cc(OC(F)F)cnc1N1C(=O)N[C@]2(CC[C@@](c3cc(F)cc(F)c3)(N(C)C)CC2)C1=O]

Cu₂O (98 mg, 0.52 mmol) and DMEDA (46 mg, 0.52 mmol) were added to a stirred solution of cis-8-(3,5-difluorophenyl)-8-(dimethylamino)-1,3-diazaspiro[4.5]decane-2,4-dione (INT_003, 0.21 g, 0.65 mmol) and 5-(difluoromethoxy)-2-iodo-3-methylpyridine (INT_034, 184 mg, 0.65 mmol) in DMA (10 mL) at room temperature in a microwave vial. The reaction mixture was stirred at 180° C. for 2 h in the microwave and subsequently cooled to room temperature, filtered through a pad of celite and washed with dichloromethane (2×20 mL). The filtrate was concentrated in vacuo and the crude product was purified by preparative HPLC to afford the title compound (35 mg, 11%) as a solid. LCMS m/z=479.3 [M−H]⁻, ¹H NMR (400 MHz, DMSO-d₆) δ: 9.09 (br, 1H), 8.31 (d, 1H), 7.76 (d, 1H), 7.36 (t, 1H), 7.20-7.14 (m, 1H), 7.11-7.08 (m, 2H), 2.51 (s, 2H), 2.14 (s, 3H), 2.07-2.02 (m, 2H), 1.95 (s, 6H), 1.86-1.78 (m, 2H), 1.65-1.60 (m, 2H).

### Pharmacological Investigations

Functional investigation on the human mu-opioid receptor (hMOP), human kappa-opioid receptor (hKOP), human delta-opioid receptor (hDOP), and human nociceptin/orphanin FQ peptide receptor (hNOP)

Human NOP Binding assay (Ardati, A., et al. (1997), Mol. Pharmacol., 51: 816-824)

The human nociceptin (hNOP) receptor binding assay was performed as an filtration-based radio agonist binding assay. Cell membrane homogenates of transfected Chem-1 cells (5 μg) were incubated for 60 min at 22° C. with 0.1 nM [3H]nociceptin in the absence or presence of the test compound in a buffer containing 50 mM Tris-HCl (pH 7.4), 5 mM MgCl2 and 0.1% BSA in a final volume of 200 μl in a 96 well plate. Nonspecific binding was determined in the presence of 1 μM nociceptin.

Test compound was added at a 100× concentrated solution in solvent and final assay DMSO concentration was 1% maximum which also served as respective vehicle control. Following incubation, the samples were filtered rapidly under vacuum through glass fiber filters (GF/B, Packard, presoaked with 0.3% PEI) and rinsed several times with ice-cold 50 mM Tris-HCl using a 96-sample cell harvester (Unifilter, Packard). The filters were dried and counted for radioactivity in a scintillation counter (Topcount, Packard) using a scintillation cocktail (Microscint 0, Packard).

The results are expressed as a percent inhibition of the control radioligand specific binding. Half-maximal inhibitory concentration (IC50) values reflecting 50% displacement of [3H]nociceptinspecific receptor binding are calculated by nonlinear regression analysis and Ki values are calculated by using the Cheng-Prusoff equation (Cheng Y. and Prusoff W. H. (1973), Biochem. Pharmacol. 22:3099-3108).

Human DOP Binding assay (Simonin, F. et al. (1994), Mol. Pharmacol., 46:1015-1021)

The human δ2-opioid (hDOP) receptor binding assay was performed as an filtration-based radio agonist binding assay. Cell membrane homogenates of transfected Chem-1 cells (1 μg) were incubated for 60 min at 22° C. with 0.5 nM [3H]DADLE in the absence or presence of the test compound in a buffer containing 50 mM Tris-HCl (pH 7.4) and 5 mM MgCl2 in a final volume of 200 μl in a 96 well plate. Nonspecific binding was determined in the presence of 10 μM naltrexone.

Test compound was added at a 100× concentrated solution in solvent and final assay DMSO concentration was 1% maximum which also served as respective vehicle control.

Following incubation, the samples were filtered rapidly under vacuum through glass fiber filters (GF/B, Packard, presoaked with 0.3% PEI) and rinsed several times with ice-cold 50 mM Tris-HCl using a 96-sample cell harvester (Unifilter, Packard). The filters were dried and counted for radioactivity in a scintillation counter (Topcount, Packard) using a scintillation cocktail (Microscint 0, Packard).

The results are expressed as a percent inhibition of the control radioligand specific binding. Half-maximal inhibitory concentration (IC50) values reflecting 50% displacement of [3H]DADLE-specific receptor binding are calculated by nonlinear regression analysis and Ki values are calculated by using the Cheng-Prusoff equation.

### Human MOP Binding

The human μ-opioid (hMOP) receptor binding assay was performed as an filtration-based radio agonist binding assay. Cell membranes of transfected CHO cells (10 μg) were incubated for 60 min at 25° C. with 0.7 nM [3H]DAMGO in the absence or presence of the test compound in a buffer containing Tris 50 mM, MgCl2 5 mM, Saponine 10 μg/ml in a final volume of 100 μl in a 96 well plate. Nonspecific binding was determined in the presence of 10 μM DAMGO.

Following incubation, the samples were filtered rapidly over filter plates (GF/C). Filters were washed six times with 0.5 ml of ice-cold washing buffer and 50 μl of Microscint 20 (Packard) was added in each well. The plates were incubated 15 min on an orbital shaker and then counted with a TopCount™ for 30 sec/well.

The results are expressed as a percent inhibition of the control radioligand specific binding. Half-maximal inhibitory concentration (IC50) values reflecting 50% displacement of [3H]DAMGO-specific receptor binding are calculated by nonlinear regression analysis and Ki values are calculated by using the Cheng-Prusoff equation.

### Human KOP Binding

The human κ-opioid (hKOP) receptor assay was performed as an filtration-based radio agonist binding assay. Cell membranes of transfected CHO cells (15 μg) were incubated for 60 min at 25° C. with 0.9 nM [3H]U-69593 in the absence or presence of the test compound in a buffer containing Tris 50 mM, MgCl2 5 mM, Saponine 10 g/ml in a final volume of 100 μl in a 96 well plate. Nonspecific binding was determined in the presence of 10 μM U-50488.

Following incubation, the samples were filtered rapidly over filter plates (GF/C). Filters were washed six times with 0.5 ml of ice-cold washing buffer and 50 μl of Microscint 20 (Packard) was added in each well. The plates were incubated 15 min on an orbital shaker and then counted with a TopCount™ for 30 sec/well.

The results are expressed as a percent inhibition of the control radioligand specific binding. Half-maximal inhibitory concentration (IC50) values reflecting 50% displacement of [3H]U-69593-specific receptor binding are calculated by nonlinear regression analysis and Ki values are calculated by using the Cheng-Prusoff equation.

Results are compiled in the table here below:

| | hNOP | hMOP | hDOP | hKOP |
|---|---|---|---|---|
| Example | Ki [μM] | Ki [μM] | Ki [μM] | Ki [μM] |
| EX-I-01 | 0.0015 | >10 | 0.6346 | 2.8329 |
| EX-I-02 | 0.0034 | >10 | 0.7425 | >10 |
| EX-I-03 | 0.0017 | >10 | 1.0003 | 3.1581 |
| EX-I-04 | 0.0013 | 3.2554 | 0.5341 | 2.1853 |
| EX-I-05 | 0.0005 | 2.5829 | 0.1964 | 1.4139 |
| EX-I-06 | 0.0044 | >10 | 2.076 | 4.9714 |
| EX-I-07 | 0.0035 | >10 | 0.5931 | 2.4688 |
| EX-I-08 | 0.0036 | 2.9856 | 1.1635 | 5.5508 |
| EX-I-09 | 0.0067 | >10 | 0.9159 | 4.5347 |
| EX-I-10 | 0.0027 | >1 | 0.7843 | 2.1789 |
| EX-I-11 | 0.0018 | >1 | 0.3825 | >1 |
| EX-I-12 | 0.0027 | >1 | 0.3427 | 3.0207 |
| EX-I-13 | 0.0042 | 3.3827 | 0.4053 | 3.1819 |
| EX-I-14 | 0.0092 | >10 | 0.5731 | 4.3603 |
| EX-I-15 | 0.0068 | >10 | 1.3911 | 2.5386 |
| EX-I-16 | 0.0091 | >10 | 0.8731 | 6.0234 |
| EX-I-17 | 0.0023 | >10 | 0.8417 | >1 |
| EX-I-18 | 0.0037 | >10 | 0.5131 | 3.6174 |
| EX-I-19 | 0.0055 | >10 | 0.3890 | 4.1785 |
| EX-I-20 | 0.0027 | >10 | 0.5868 | 3.8706 |
| EX-I-21 | 0.0044 | >10 | 0.9667 | 6.5233 |
| EX-I-22 | 0.0029 | >10 | 1.0089 | 7.3610 |
| EX-I-23 | 0.0019 | >10 | 0.8694 | 0.4330 |
| EX-I-24 | 0.0008 | >1 | 0.4825 | 1.6365 |
| EX-I-25 | 0.0036 | >10 | 0.8732 | 6.6554 |
| EX-I-26 | 0.0024 | 4.2394 | 0.6270 | 4.7755 |
| EX-I-27 | 0.0007 | 1.6716 | 0.7374 | 1.1412 |
| EX-I-28 | 0.0013 | 2.8314 | 0.9290 | >10 |

### Comparative Examples

| EX-C-[ . . . ]: | | | | |
|---|---|---|---|---|
| X = -H (comparative) | | | | |
| EX-I-[ . . . ] | | hNOP | hDOP | hDOP/hNOP |
| X = -F (inventive) | | Ki [μM] | Ki μM] | selectivity ratio |
| EX-C-01 EX-I-01 | 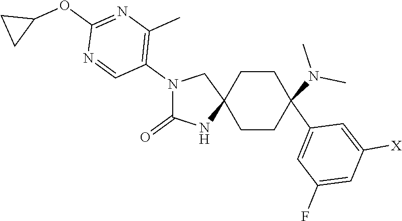 | 0.0012 0.0015 | 0.1515 0.6346 | 126 423 |
| EX-C-02 EX-1-02 | 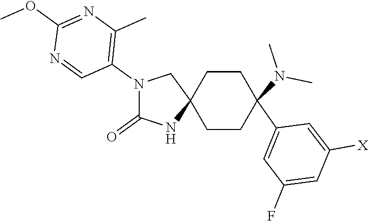 | 0.0046 0.0034 | 0.1539 0.7425 | 33 218 |
| EX-C-03 EX-1-03 | 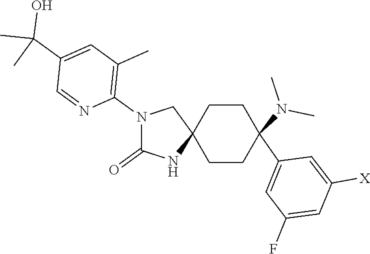 | 0.0012 0.0017 | 0.1940 1.0003 | 162 588 |
| EX-C-04 EX-1-04 | 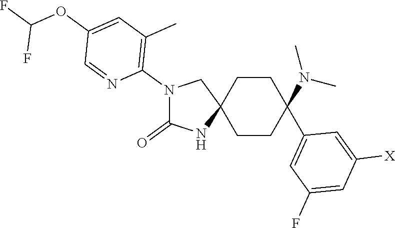 | 0.0011 0.0013 | 0.1406 0.5341 | 128 411 |
| Ex-C-05 EX-1-05 | 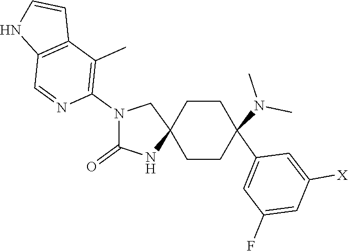 | 0.0011 0.0005 | 0.0568 0.1964 | 52 393 |
| EX-C-06 EX-I-06 | 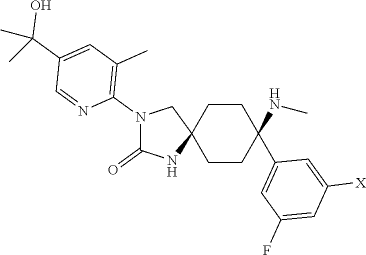 | 0.0059 0.0044 | 1.167 2.076 | 198 472 |
| EX-C-07 EX-1-07 | 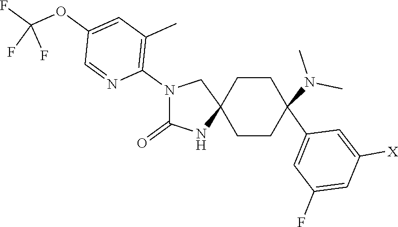 | 0.0038 0.0035 | 0.2577 0.5931 | 68 169 |
| EX-C-08 EX-1-08 | 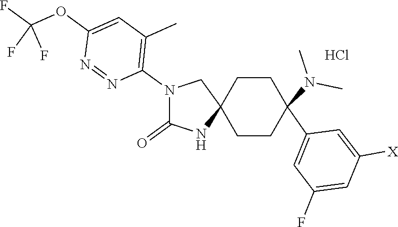 | 0.0091 0.0036 | 0.7304 1.1635 | 80 323 |
| EX-C-09 EX-1-09 | 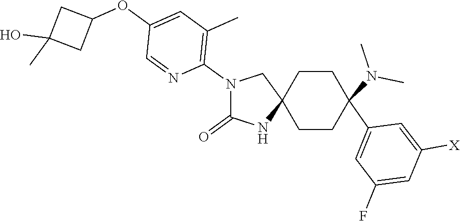 | 0.0036 0.0067 | 0.3006 0.9159 | 84 137 |
| EX-C-10 EX-1-10 | 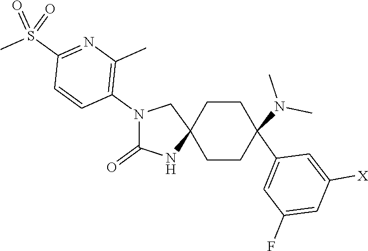 | 0.0032 0.0027 | 0.1071 0.7843 | 33 290 |
| EX-C-11 EX-I-11 | 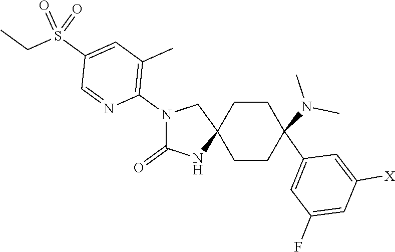 | 0.0023 0.0018 | 0.2316 0.3825 | 101 213 |
| EX-C-12 EX-I-12 | 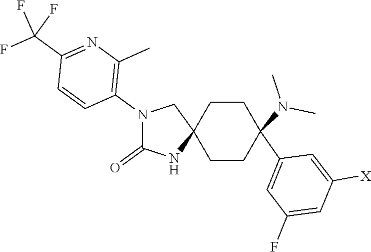 | 0.0039 0.0027 | 0.1346 0.3427 | 34 127 |
| EX-C-13 EX-I-13 | 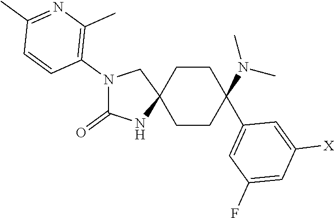 | 0.0075 0.0042 | 0.1359 0.4053 | 18 97 |
| EX-C-14 EX-I-14 | 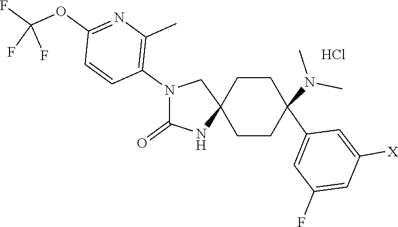 | 0.0295 0.0092 | 0.2122 0.5731 | 7 62 |
| EX-C-15 EX-I-15 | 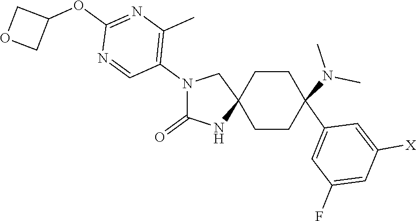 | 0.0051 0.0068 | 0.4652 1.3911 | 91 205 |
| EX-C-16 EX-I-16 | 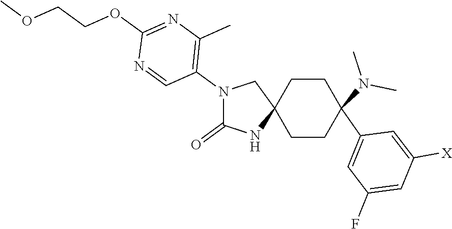 | 0.0088 0.0091 | 0.3376 0.8731 | 38 96 |
| EX-C-17 EX-I-17 | 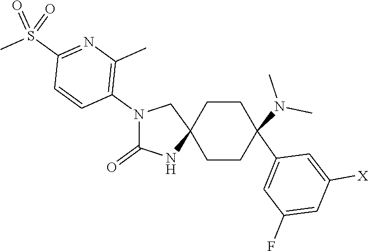 | 0.0046 0.0023 | 0.3654 0.8417 | 79 366 |
| EX-C-18 EX-I-18 | 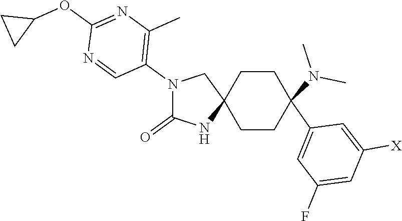 | 0.0030 0.0037 | 0.2426 0.5131 | 81 139 |
| EX-C-19 EX-I-19 | 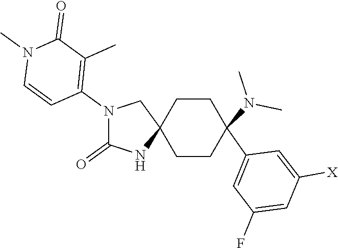 | 0.0068 0.0055 | 0.1907 0.3890 | 28 71 |
| EX-C-20 EX-I-20 | 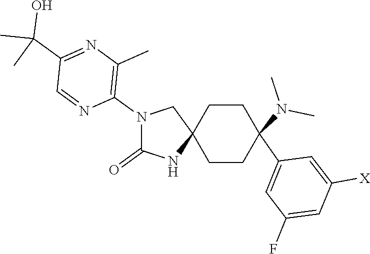 | 0.0037 0.0027 | 0.3054 0.5868 | 83 217 |
| EX-C-21 EX-I-21 | 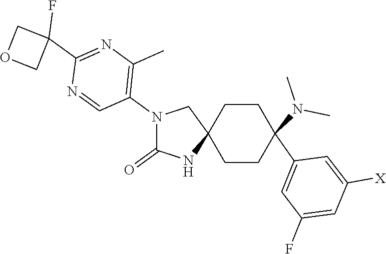 | 0.0034 0.0044 | 0.4381 0.9667 | 129 220 |
| EX-C-22 EX-I-22 | 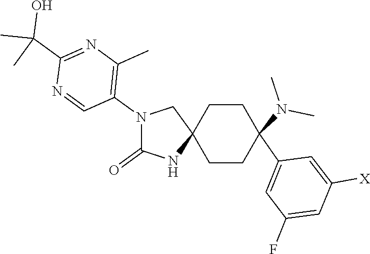 | 0.0030 0.0029 | 0.4557 1.0089 | 152 348 |
| EX-C-23 EX-I-23 | 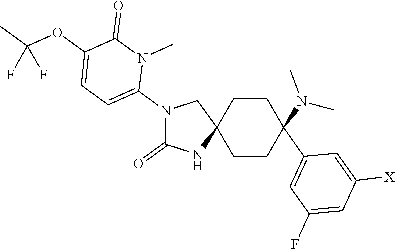 | 0.0023 0.0019 | 0.5979 0.8694 | 260 458 |
| EX-C-24 EX-I-24 | 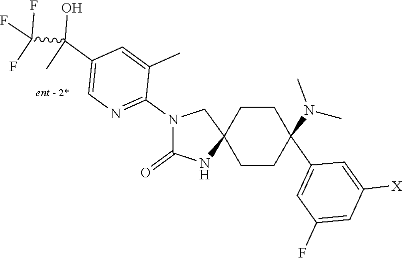 * second eluting single enantiomer | 0.0006 0.0008 | 0.2079 0.4825 | 347 603 |
| EX-C-25 EX-I-25 | 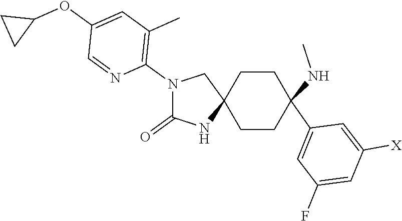 | 0.0071 0.0036 | 0.6472 0.8732 | 91 243 |
| EX-C-26 EX-I-26 | 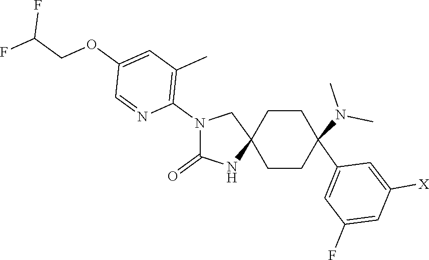 | 0.0024 0.0024 | 0.3045 0.6270 | 127 261 |
| EX-C-27 EX-I-27 | 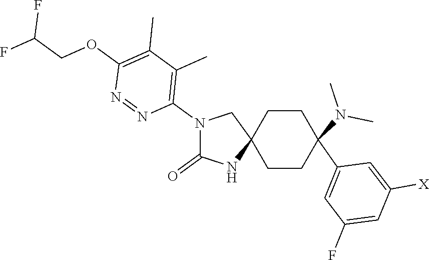 | 0.0010 0.0007 | 0.2833 0.7374 | 283 1053 |
| EX-C-28 EX-I-28 | 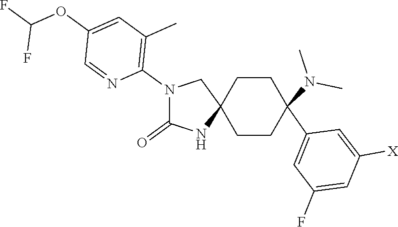 | 0.0015 0.0013 | 0.2601 0.9290 | 173 715 |

1. A compound according to general formula (I) 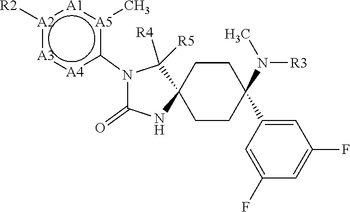
wherein
A1 represents N or CR1;
A2 represents N or C;
A3 represents N or CH;
A4 represents N or CH;
A5 represents N or C;
with the proviso that one or two of A1, A2, A3, A4 and A5 mean N, whereas the remaining three or four of A1, A2, A3, A4, and A5 do not mean N;
R1 represents —H, —CH₃ or ═O;
R2 represents
—C₁-C₆-alkyl, linear or branched, saturated or unsaturated, unsubstituted or substituted with one, two, three or four substituents independently selected from —F and —OH;
—C₃-C₇-heterocycloalkyl, saturated or unsaturated, unsubstituted or substituted with one, two, three or four substituents independently selected from —F, —OH and —CH₃;
—S(O)₂—C₁-C₄-alkyl, linear, branched or cyclic, unsubstituted or substituted with one, two or three substituents —F;
—O—C₁-C₆-alkyl, linear or branched, saturated or unsaturated, unsubstituted or substituted with one, two, three or four substituents independently selected from —F, —OH and —OCH₃;
—O—C₃-C₈-cycloalkyl, saturated or unsaturated, unsubstituted or substituted with one, two, three or four substituents independently selected from —F, —OH and —CH₃; or
—O—C₃-C₇-heterocycloalkyl, saturated or unsaturated, unsubstituted or substituted with one, two, three or four substituents independently selected from —F and —OH;
or R1 and R2 together with A1 and A2 form a ring and mean —CH═CN—NH—;
R3 represents —H or —CH₃;
R4 and R5 both represent either each —H or together mean ═O;
or a physiologically acceptable salt thereof.

2. The compound according to claim 1, wherein
(i) one of A1, A2, A3, A4 and A5 represents N, whereas the remaining four of A1, A2, A3, A4, and A5 do not represent N; or
(ii) two of A1, A2, A3, A4 and A5 represent N, whereas the remaining three of A1, A2, A3, A4, and A5 do not represent N.

3. The compound according to claim 1, wherein A1 represents CR1; A2 represents C; A3 represents CH; A4 represents N; and A5 represents C.

4. The compound according to claim 1, wherein A1 represents N; A2 represents C; A3 represents CH; A4 represents CH; and A5 represents C

5. The compound according to claim 1, wherein A1 represents CR1; A2 represents C; A3 represents N; A4 represents N; and A5 represents C.

6. The compound according to claim 1, wherein A1 represents N; A2 represents C; A3 represents N; A4 represents CH; and A5 represents C.

7. The compound according to claim 1, wherein A1 represents CR1 with R1 meaning ═O; A2 represents N; A3 represents CH; A4 represents CH; and A5 represents C.

8. The compound according to claim 1, wherein A1 represents N; A2 represents C; A3 represents CH; A4 represents N; and A5 represents C.

9. The compound according to claim 1, wherein A1 represents CR1 with R1 meaning ═O; A2 represents C; A3 represents CH; A4 represents CH; and A5 represents N.

10. The compound according to claim 1, wherein R1 (if present) represents —H, or —CH₃.

11. The compound according to claim 1, wherein R2 is selected from —CH₃, —CF₃, —C(CH₃)₂OH, and —C(CH₃)(OH)CF₃.

12. The compound according to claim 1, wherein R2 represents 3-fluorooxetan-3-yl.

13. The compound according to claim 1, wherein R2 is selected from —S(O)₂CH₃ and —S(O)₂CH₂CH₃.

14. The compound according to claim 1, wherein R2 is selected from —OCH₃, —OCHF₂, —OCH₂CHF₂, —OCF₂CH₃, —OCF₃, —OCH₂CH₂OCH₃, —O— cyclopropyl, and 3-hydroxy-3-methylcyclobutoxy.

15. The compound according to claim 1, wherein R2 represents oxetan-3-yloxy.

16. The compound according to claim 1, wherein R1 and R2 together with A1 and A2 form a ring and mean —CH═CN—NH—.

17. The compound according to claim 1, wherein R3 represents —CH₃.

18. The compound according to claim 1, wherein R4 and R5 both mean —H.

19. The compound according to claim 1, which is selected from the group consisting of:
EX-I-01 cis-3-(5-cyclopropoxy-3-methylpyridin-2-yl)-8-(3,5-difluorophenyl)-8-(dimethylamino)-1,3-diazaspiro[4.5]decan-2-one;
EX-I-02 cis-8-(3,5-difluorophenyl)-8-(dimethylamino)-3-(2-methoxy-4-methylpyrimidin-5-yl)-1,3-diazaspiro[4.5]decan-2-one;
EX-I-03 cis-8-(3,5-difluorophenyl)-8-(dimethylamino)-3-(5-(2-hydroxypropan-2-yl)-3-methylpyridin-2-yl)-1,3-diazaspiro[4.5]decan-2-one;
EX-I-04 cis-3-(5-(difluoromethoxy)-3-methylpyridin-2-yl)-8-(3,5-difluorophenyl)-8-(dimethylamino)-1,3-diazaspiro[4.5]decan-2-one;
EX-I-06 cis-8-(3,5-difluorophenyl)-3-(5-(2-hydroxypropan-2-yl)-3-methylpyridin-2-yl)-8-(methylamino)-1,3-diazaspiro[4.5]decan-2-one;
EX-I-07 cis-8-(3,5-difluorophenyl)-8-(dimethylamino)-3-(3-methyl-5-(trifluoromethoxy)pyridin-2-yl)-1,3-diazaspiro[4.5]decan-2-one;
EX-I-08 cis-8-(3,5-difluorophenyl)-8-(dimethylamino)-3-(4-methyl-6-(trifluoromethoxy)pyridazin-3-yl)-1,3-diazaspiro[4.5]decan-2-one;
EX-I-09 cis-8-(3,5-difluorophenyl)-8-(dimethylamino)-3-(5-(3-hydroxy-3-methylcyclobutoxy)-3-methylpyridin-2-yl)-1,3-diazaspiro[4.5]decan-2-one;
EX-I-10 cis-8-(3,5-difluorophenyl)-8-(dimethylamino)-3-(3-methyl-5-(methylsulfonyl)pyridin-2-yl)-1,3-diazaspiro[4.5]decan-2-one;
EX-I-11 cis-8-(3,5-difluorophenyl)-8-(dimethylamino)-3-(5-(ethylsulfonyl)-3-methylpyridin-2-yl)-1,3-diazaspiro[4.5]decan-2-one;
EX-I-12 cis-8-(3,5-difluorophenyl)-8-(dimethylamino)-3-(2-methyl-6-(trifluoromethyl)pyridin-3-yl)-1,3-diazaspiro[4.5]decan-2-one;
EX-I-13 cis-8-(3,5-difluorophenyl)-8-(dimethylamino)-3-(2,6-dimethylpyridin-3-yl)-1,3-diazaspiro[4.5]decan-2-one;
EX-I-14 cis-8-(3,5-difluorophenyl)-8-(dimethylamino)-3-(2-methyl-6-(trifluoromethoxy)pyridin-3-yl)-1,3-diazaspiro[4.5]decan-2-one;
EX-I-15 cis-8-(3,5-difluorophenyl)-8-(dimethylamino)-3-(4-methyl-2-(oxetan-3-yloxy)pyrimidin-5-yl)-1,3-diazaspiro[4.5]decan-2-one;
EX-I-16 cis-8-(3,5-difluorophenyl)-8-(dimethylamino)-3-(2-(2-methoxyethoxy)-4-methylpyrimidin-5-yl)-1,3-diazaspiro[4.5]decan-2-one;
EX-I-17 cis-8-(3,5-difluorophenyl)-8-(dimethylamino)-3-(2-methyl-6-(methylsulfonyl)pyridin-3-yl)-1,3-diazaspiro[4.5]decan-2-one;
EX-I-18 cis-3-(2-cyclopropoxy-4-methylpyrimidin-5-yl)-8-(3,5-difluorophenyl)-8-(dimethylamino)-1,3-diazaspiro[4.5]decan-2-one;
EX-I-19 cis-8-(3,5-difluorophenyl)-3-(1,3-dimethyl-2-oxo-1,2-dihydropyridin-4-yl)-8-(dimethylamino)-1,3-diazaspiro[4.5]decan-2-one;
EX-I-20 cis-8-(3,5-difluorophenyl)-8-(dimethylamino)-3-(5-(2-hydroxypropan-2-yl)-3-methylpyrazin-2-yl)-1,3-diazaspiro[4.5]decan-2-one;
EX-I-21 cis-8-(3,5-difluorophenyl)-8-(dimethylamino)-3-(2-(3-fluorooxetan-3-yl)-4-methylpyrimidin-5-yl)-1,3-diazaspiro[4.5]decan-2-one;
EX-I-22 cis-8-(3,5-difluorophenyl)-8-(dimethylamino)-3-(2-(2-hydroxypropan-2-yl)-4-methylpyrimidin-5-yl)-1,3-diazaspiro[4.5]decan-2-one;
EX-I-23 cis-3-(5-(1,1-difluoroethoxy)-1-methyl-6-oxo-1,6-dihydropyridin-2-yl)-8-(3,5-difluorophenyl)-8-(dimethylamino)-1,3-diazaspiro[4.5]decan-2-one;
EX-I-24 cis-8-(3,5-difluorophenyl)-8-(dimethylamino)-3-(3-methyl-5-((S)-1,1,1-trifluoro-2-hydroxypropan-2-yl)pyridin-2-yl)-1,3-diazaspiro[4.5]decan-2-one;
EX-I-25 cis-3-(5-cyclopropoxy-3-methylpyridin-2-yl)-8-(3,5-difluorophenyl)-8-(methylamino)-1,3-diazaspiro[4.5]decan-2-one;
EX-I-26 cis1-3-(5-(2,2-difluoroethoxy)-3-methylpyridin-2-yl)-8-(3,5-difluorophenyl)-8-(dimethylamino)-1,3-diazaspiro[4.5]decan-2-one;
EX-I-27 cis-3-(6-(2,2-difluoroethoxy)-4,5-dimethylpyridazin-3-yl)-8-(3,5-difluorophenyl)-8-(dimethylamino)-1,3-diazaspiro[4.5]decan-2-one;
EX-I-28 cis-3-(5-(difluoromethoxy)-3-methylpyridin-2-yl)-8-(3,5-difluorophenyl)-8-(dimethylamino)-1,3-diazaspiro[4.5]decane-2,4-dione;
and the physiologically acceptable salts thereof.

20. A pharmaceutical composition comprising a compound according to claim 1.

21. A method of treating pain in a patient in need thereof, said method comprising administering to said patient an effective amount therefor of a compound according to claim 1.
